(19) United States
(12) Patent Application Publication
(10) Pub. No.: US 2023/0160025 A1
(43) Pub. Date: May 25, 2023

(54) POINT-OF-CARE SARS-COV-2 VIRUS DIAGNOSTIC DEVICE AND METHODS OF USE THEREOF

(71) Applicant: GoDx, Inc., Madison, WI (US)

(72) Inventors: Chang Hee KIM, Waunakee, WI (US); Jacob Jerome TOKAR, Madison, WI (US); Fernando R. MOREIRA, Madison, WI (US); Xiao JIANG, Madison, WI (US)

(21) Appl. No.: 18/049,552

(22) Filed: Oct. 25, 2022

Related U.S. Application Data
(63) Continuation-in-part of application No. PCT/US2021/035977, filed on Jun. 4, 2021.
(60) Provisional application No. 63/034,710, filed on Jun. 4, 2020.
(60) Provisional application No. 63/079,828, filed on Sep. 17, 2020.
(60) Provisional application No. 63/271,521, filed on Oct. 25, 2021.

(51) Int. Cl. C12Q 1/70

(52) U.S. Cl. CPC C12Q 1/701

(57) ABSTRACT

The present invention provides compositions comprising primers for loop-mediated isothermal amplification of a target nucleotide sequence of SARS-CoV-2, as well as point-of-care diagnostic devices and kits comprising said compositions. Methods of using the compositions, devices, and kits to detect SARS-CoV-2 in a sample are also provided.

### CROSS-REFERENCE TO RELATED APPLICATIONS

This application is a continuation-in-part of International Application No. PCT/US2021/035977, filed on Jun. 4, 2021, which claims priority to U.S. Provisional Application No. 63/034,710, filed on Jun. 4, 2020, U.S. Provisional Application No. 63/079,828, filed on Sep. 17, 2020, and U.S. Provisional Application No. 63/271,521, filed on Oct. 25, 2021, the contents of each are incorporated by reference in their entireties.

### STATEMENT REGARDING FEDERALLY SPONSORED RESEARCH

This invention was made with government support under R44TR001912 awarded by National Center for Advancing Translational Sciences at the National Institutes of Health. The government has certain rights in the invention.

### SEQUENCE LISTING

A Sequence Listing accompanies this application and is submitted as an XML file of the sequence listing named “165369.00035.xml” which is 66,492 bytes in size and was created on Oct. 10, 2022. The sequence listing is electronically submitted via Patent Center with the application and is incorporated herein by reference in its entirety.

### INTRODUCTION

The coronavirus disease (COVID-19) pandemic, caused by the virus SARS-CoV-2, has exposed major vulnerabilities in our nation’s healthcare system, including its inability to quickly develop and administer rapid and accurate tests for detection of the infectious agent. The first Food and Drug Administration (FDA)-approved tests administered by the Centers of Disease Control and Prevention (CDC) and the New York State Department of Health were based on reverse transcription polymerase chain reaction (RT-PCR). These tests are unacceptably slow and expensive in view of the rapid spread of the virus. Because they require that samples are sent away to a laboratory for analysis, it takes 3 to 5 days from the time they are requested to receive results. Additionally, these tests require 6 hours of extensive labor and expensive instruments that are limited in availability. Further, they require the use of a slow (20 minute), labor-intensive (5 steps), and expensive RNA extraction procedure that creates bottlenecks in the testing pipeline. In total, such RT-PCR tests could cost up to $1200.00 per patient.

Despite the progress that has been made in vaccinating against this virus, more diagnostic tests will be required to identify SARS-CoV-2 infected individuals, especially those that are asymptomatic. In areas that have not been reached by vaccination efforts, there is still a dire need to have diagnostic tests readily available to facilitate rapid and effective quarantining of the infected individuals. Ideally, the tests should be made available to people with any type of respiratory symptoms and to pre-symptomatic or asymptomatic individuals who have encountered confirmed COVID-19 patients. Thus, there remains a need for improved, inexpensive diagnostic tests for SARS-CoV-2 that provide rapid results.

### SUMMARY

Disclosed herein are compositions, point-of-care diagnostic devices, kits, and methods for the detection of SARS-CoV-2.

One aspect of the invention is a composition comprising primers for loop-mediated isothermal amplification of a target nucleotide sequence of SARS-CoV-2.

In some embodiments, the composition comprises a F3 primer comprising nucleotide sequence GATTTTTGTGGAAAGGGCTATC (SEQ ID NO: 1) or TTTTTGTGGAAAGGGCTATC (SEQ ID NO: 2) (5′ - 3′); a B3 primer comprising nucleotide sequence CAAACCAGTGTGTGCCAT (SEQ ID NO: 3) (5′ - 3′); a FIP primer comprising a F1c nucleotide sequence AGGGACATAAGTCACATGCAAGAA (SEQ ID NO: 4) and a F2 nucleotide sequence TTCTTATGTCCTTCCCTCAGT (SEQ ID NO: 5) and a spacer therebetween (5′ - 3′); and a BIP primer comprising a B1c nucleotide sequence AGAAAAGAACTTCACAACTGCTCC (SEQ ID NO: 6) and a B2 nucleotide sequence CAAAGACACCTTCACGAGG (SEQ ID NO: 7) and a spacer therebetween (5′ - 3′). In some embodiments, the composition further comprises a loop forward primer comprising nucleotide sequence GACTACACCATGAGGTGCTG (SEQ ID NO: 8) (5′ - 3′) and a loop backward primer comprising nucleotide sequence CATTTGTCATGATGGAAAAG (SEQ ID NO: 9) (5′ -3′). In some embodiments, the target nucleotide sequence comprises SEQ ID NO: 69.

In other embodiments, the composition comprises a F3 primer comprising nucleotide sequence TTGGTGCAGGTATATGCG (SEQ ID NO: 12) (5′ - 3′); a B3 primer comprising nucleotide sequence ACATTGTACAATCTACTGATGTC (SEQ ID NO: 13) (5′ - 3′); a FIP primer comprising a F1c nucleotide sequence TAGGCAATGATGGATTGACTAGCTA (SEQ ID NO: 14), a F2 nucleotide sequence TTATCAGACTCAGACTAATTCTCC (SEQ ID NO: 15), and a spacer therebetween (5′ - 3′); and a BIP primer comprising a B1c nucleotide sequence AACTCTATTGCCATACCCACAAAT (SEQ ID NO: 16), a B2 nucleotide sequence TTGGTCATAGACACTGGTAG (SEQ ID NO: 17), and a spacer therebetween (5′ -> 3′). In some embodiments, the composition further comprises a loop forward primer comprising nucleotide sequence CACTACGTGCCCGCCGA (SEQ ID NO: 18) (5′ - 3′) and a loop backward primer comprising nucleotide sequence TTTACTATTAGTGTTACC (SEQ ID NO: 19) (5′ - 3′). In some embodiments, the target nucleotide sequence comprises SEQ ID NO: 70.

In still other embodiments, the composition comprises a F3 primer comprising nucleotide sequence TTGGTGCAGGTATATGCG (SEQ ID NO: 12) (5′ - 3′); a B3 primer comprising nucleotide sequence ACATTGTACAATCTACTGATGTC (SEQ ID NO: 13) (5′ - 3′); a FIP primer comprising a F1c nucleotide sequence TAGGCAATGATGGATTGACTAGCTA (SEQ ID NO: 14), a F2 nucleotide sequence TTATCAGACTCAGACTAATTCTCG (SEQ ID NO: 72), and a spacer therebetween (5′ - 3′); and a BIP primer comprising a B1c nucleotide sequence AACTCTATTGCCATACCCACAAAT (SEQ ID NO: 16), a B2 nucleotide sequence TTGGTCATAGACACTGGTAG (SEQ ID NO: 17), and a spacer therebetween (5′ -> 3′). In some embodiments, the composition further comprises a loop forward primer comprising nucleotide sequence CACTACGTGCCCGCCGA (SEQ ID NO: 18) (5′ - 3′) and a loop backward primer comprising nucleotide sequence TTTACTATTAGTGTTACC (SEQ ID NO: 19) (5′ - 3′). In some embodiments, the target nucleotide sequence comprises SEQ ID NO: 73.

A second aspect of the invention is a nucleic acid prepared using any of the primers disclosed herein.

A third aspect of the invention is a method for detecting SARS-CoV-2. The method may comprise contacting any of the primers or compositions disclosed herein with a sample under conditions sufficient for loop-mediated isothermal amplification of the target nucleotide sequence of SARS-CoV-2 and detecting the presence or absence of an amplification product of the target nucleotide sequence.

A fourth aspect of the invention is a diagnostic device for detecting SARS-CoV-2. The diagnostic device comprises at least one reaction chamber comprising any of the primers or compositions disclosed herein and a detection device that provides a readout that indicates whether the target nucleic acid is present in the sample.

A fifth aspect of the invention is a kit for detecting SARS-CoV-2. The kit may comprise any of the primers or compositions disclosed herein.

### BRIEF DESCRIPTION OF THE DRAWINGS

FIG. 1 is a schematic of the diagnostic method described herein.

FIG. 2 is a schematic of reverse transcription loop-mediated isothermal amplification (RT-LAMP).

FIG. 3 is a plot of RT-LAMP amplification of synthetic RNA target sequences supplied at 125 or 250 copies per reaction. NTC: non-template control.

FIG. 4 shows the readout of a lateral flow assay performed following RT-LAMP amplification of synthetic RNA target sequences supplied at 200 or 1000 copies per reaction. NTC: non-template control.

FIG. 5 is a schematic of an exemplary point-of-care diagnostic device. This design includes a lysis cartridge that is used to lyse the sample, forming a lysate solution. The nucleic acid binding stage (Slider 1) is initially positioned directly beneath the sample inlet so it can collect and bind nucleic acids from the lysate solution. The lysate seeps through a nucleic acid binding substrate positioned at the bottom of a funnel built into Slider 1. The substrate binds nucleic acids and filters out large particulate. The user then slides Slider 1 from position one — beneath the sample inlet —to position two. This action may pierce a blister pouch holding wash buffer, which flows through the substrate of Slider 1, washing the bound nucleic acids. A waste pad beneath Slider 1 absorbs the lysis and wash buffers passing through the nucleic acid binding substrate during both of these initial steps. Next, Slider 1 is slid from position two to position three where it may pierce a blister pouch containing elution buffer. In position three, Slider 1 is positioned over the eluate inlet of the amplification stage (Slider 2) in such a way that when the elution buffer flows through the substrate of Slider 1, it delivers the eluate to the eluate inlet of Slider 2. The eluate then flows through microchannels to reaction chambers of Slider 2, which contain a nucleic acid amplification reagent. In this embodiment, three of the reaction chambers contain the necessary enzymes and primers to amplify diagnostic markers that are indicative of the presence of a pathogen, and the fourth chamber contains primers to amplify human Amylase, ActB, or RNAseP as a control. Amplification is allowed to proceed for approximately 15-20 minutes. Then, the user moves Slider 2 from its initial position to its final position where Slider 2 may pierce a blister pouch containing lateral flow assay (LFA) running buffer. The running buffer forces the amplicons to flow from the reaction chambers through microchannels that converge at the output port of Slider 2, positioned directly beneath the collection pad of the diagnostic device (LFA). When the output droplet merges with the LFA pad, capillary flow within the LFA drives further flow of the amplicons from Slider 2 through the LFA for pathogen detection.

FIG. 6 is a schematic of an exemplary point-of-care kit. The platform consists of a sample collection tube, a transfer pipette or swab, a lysis cartridge, a disposable processing/detection device, and a reusable handheld controller.

FIG. 7 is a schematic of an exemplary lysis cartridge. The lysis cartridge may be configured to filter out particulate and chemical inhibitors in order to improve nucleic acid recovery and amplification.

FIG. 8 is a schematic of an exemplary nucleic acid binding stage. The stage consists of a collection funnel, an outlet where the nucleic acid binding substrate is affixed, and a sponge ramp that compresses and drains reagent sponges.

FIG. 9 is a schematic of an exemplary nucleic acid purification device that utilizes laminated glass microspheres. (a): Schematic diagram of the testing design and lamination process. The lamination process is performed by sandwiching the microspheres between two glass fiber membranes. The glass fiber membrane-microspheres sandwich is then sandwiched between two pieces of thermoplastic film. The final layered assembly is laminated by passing through a heat laminator. The laminated microspheres are adhered to the bottom of the 3D-printed funnel for testing nucleic acid isolation. (b): A 3D-printed funnel with laminated borosilicate glass microspheres attached to the opening via a piece of double-sided adhesive. (c): A photograph demonstrating analyte filtration using food dye.

FIG. 10 is a schematic depicting reagent delivery using sponges. When a sample is being processed, the shelf-life reagent reservoirs are pierced and drained into sponges for temporary storage as they await their turn in the protocol sequence. The sponge ramp gently compresses the sponges, draining reagents into the funnel of the binding stage.

FIG. 11 is a schematic of an exemplary protocol in which the point-of-care diagnostic device is used to analyze a sample. (a): The sample is lysed in the lysis cartridge, which is a pierce-bottom tube. (b): The bottom of the lysis cartridge is pierced as it is inserted into the device, draining the lysate into the processing funnel. The binding membrane attached to the funnel output binds nucleic acids as the lysate drains through. Wash and elution buffers are subsequently drained through the membrane, with the eluate wicking into a microchannel (paper or capillary) rather than the waste pad. (c): Eluate wicks through the microchannel into the reaction chamber. (d): Nucleic acids and reaction reagents are mixed when rehydration buffer is added to the chamber. (e): An amplification reaction begins when the chamber is heated. (f): Amplicons are wicked through another microchannel into the sample pad. (g): Running buffer is released from a reagent blister pouch, driving amplicons through to the LFA beyond.

FIG. 12 is a schematic depicting how the diagnostic platform can be adapted to a direct-to-amplification protocol. (a): The sample is mixed in dilution buffer prior to heat lysis. The heat used for sample lysis is initiated when the protocol is started in the instrument program. This and subsequent automated steps follow a specific protocol timeline that is programed into the software. (b): Following heat lysis, the lysis cartridge is inserted into the sample inlet of the diagnostic device, draining the lysate into a microchannel (paper or capillary). The lysate wicks along the microchannel into the reaction chamber. (c): The rehydration blister pouch is burst and drained into the reaction chamber, which is then heated to initiate the amplification reaction. The timing of reaction heating is pre-determined by the protocol program. The progression of lysate fluid and rehydration of reaction reagents is known to take a specific amount of time, which will be represented in the timing of the automated program. (d): The reaction chamber is connected to another microchannel via a magnetic valve, and wicks amplicons towards the sample pad of the lateral flow assay. The magnetic valve is actuated by a magnet housed in the handheld instrument. One mode of magnetic valve actuation is to mount a magnet to a lead screw connected to a stepper motor. Being highly controllable and precise, the movements of the stepper motor-lead screw mechanism will occur at specifically programed times built into the instrument software. Alternatively, a sequence of automated electromagnets can be used in place of a magnet mounted to a stepper motor-lead screw mechanism. The running buffer blister is then burst, driving amplicons through the detection strip for the user to read and interpret results.

FIG. 13 shows photographs of an exemplary diagnostic device. (a): The device is assembled from 3D printed parts, paper microchannels, and a LFA strip. (b): The assembled prototype draws in sample lysate through the inlet into the reaction chamber, while the sliding channel is positioned at the far end of its slot. (c): Sample is added to the inlet port. (d): Sample lysate wicks through the microchannel into the reaction chamber, providing RT-LAMP template. (e): The sliding channel is moved into the active position (yellow arrow), connecting the reaction chamber to the secondary microchannel. (f): Amplicons wick through the secondary channel and into the LFA strip, providing a positive or negative readout.

FIG. 14 illustrates the interpretation of lateral flow assay results. Examples of positive, negative, and invalid results are shown.

FIG. 15 shows examples of T and C lines generated by a positive control (left) and a negative control (right) on LFA strips.

FIG. 16 is a schematic of the diagnostic kit workflow described in Example 6.

FIG. 17 shows the lateral flow assay readout following RT-LAMP amplification of various amounts of SARS-CoV-2 delta variant RNA. Two lines on the LFA strip indicate a positive result, whereas one line indicates a negative result. Assays H1-H3 contain 2000 RNA copies/µl, H4-H6 contain 666 copies/µl, H7-H9 contain 222 copies/µl, and H10-H12 contain 0 copies/µl.

### DETAILED DESCRIPTION

In the present application, the inventors provide a novel diagnostic test for SARS-CoV-2. The test is simple, rapid, and low-cost. This test can provide results within a 35-minute visit to the doctor’s office, allowing for faster isolation and treatment of infected patients. In this test, viral RNA is amplified using reverse transcription loop-mediated isothermal amplification (RT-LAMP), and is detected using an amplicon detection assay, such as a lateral flow assay (LFA). RT-LAMP is an isothermal amplification method that is performed at a constant temperature (i.e., 60-65° C.). Thus, the diagnostic test of the present invention can be performed using (1) a simple water bath or heat-block or (2) a point-of-care device fitted with a heating element.

Importantly, because this test does not require the use of an expensive thermal cycler instrument, it can be performed in a wider range of settings than conventional PCR-based methods. For example, the test can be performed in community hospitals, primary care offices, mobile clinics, and simple physician’s office laboratories (POL) that are equipped with a with a basic water bath or heater. When the test is incorporated into a point-of-care device, it may also be run in settings such as homes, nursing homes, workplaces, meat processing plants, prisons, and rapid screening centers without sending samples to a centralized laboratory. Thus, this test will be particularly valuable in resource-limited settings in the developing world.

The diagnostic test of the present invention is also high throughput since multiple tests can be set up and run without waiting for instruments to finish running a limited number of samples. The test can be run as needed, without waiting to batch samples for loading into an instrument. Thus, this technology should immediately increase the overall testing capacity of a facility because there is no lead time for purchasing instruments.

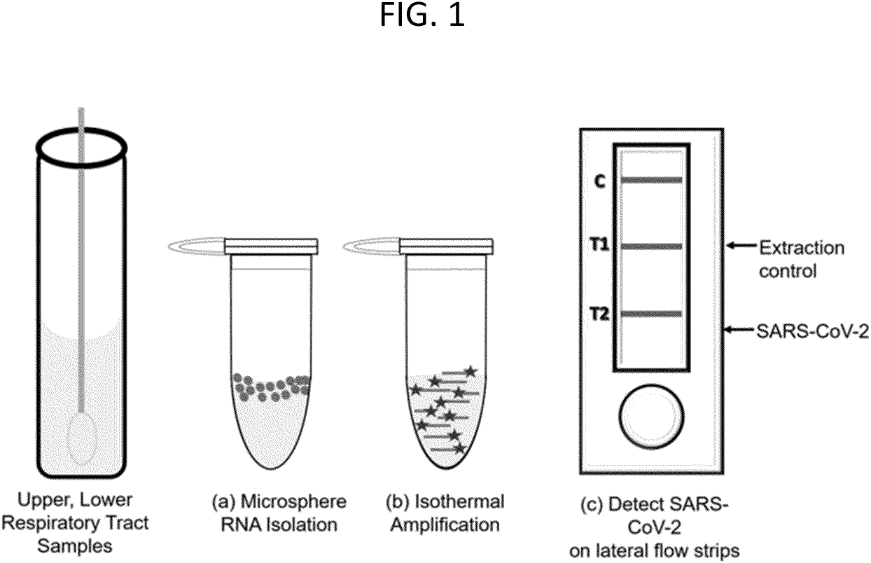

Optionally, the diagnostic test may further comprise an RNA purification and/or isolation step, which may be performed using microspheres. See FIG. 1. However, the test may also be performed directly on upper respiratory specimens, such as nasopharyngeal (NP) swabs, without RNA extraction, which allows this technology to side-step shortages in RNA extraction kits. Direct testing without RNA extraction is made possible by inactivation of RNases in the sample, e.g., using an inactivation buffer and heat lysis of the virus.

### Reverse Transcriptase Loop-Mediated Isothermal Amplification (RT-LAMP)

RT-LAMP is a one-step nucleic acid amplification method that is commonly used for the identification of pathogens. RT-LAMP uses reverse transcriptase to synthesize complementary DNA (cDNA) from RNA sequences and to amplify the sequences for detection. The cDNA is then efficiently amplified using DNA polymerase, generating roughly 10⁹ copies per hour.

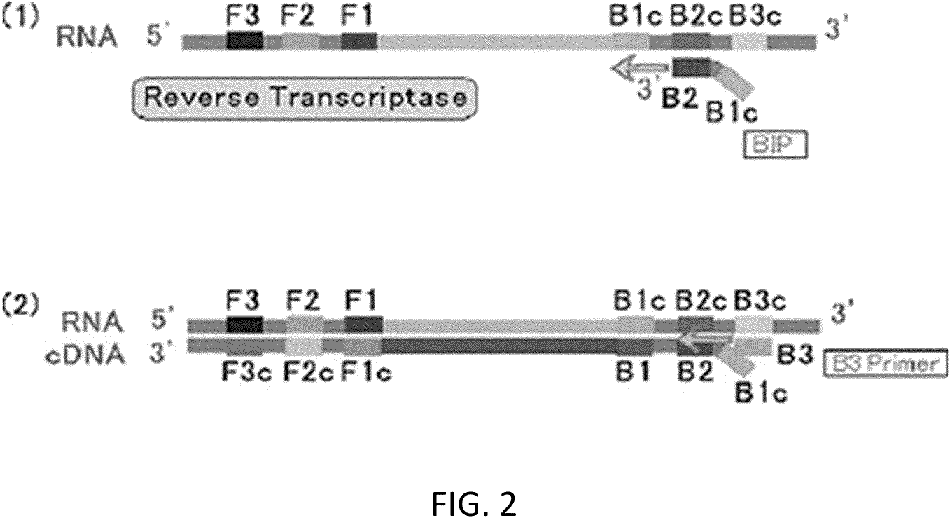

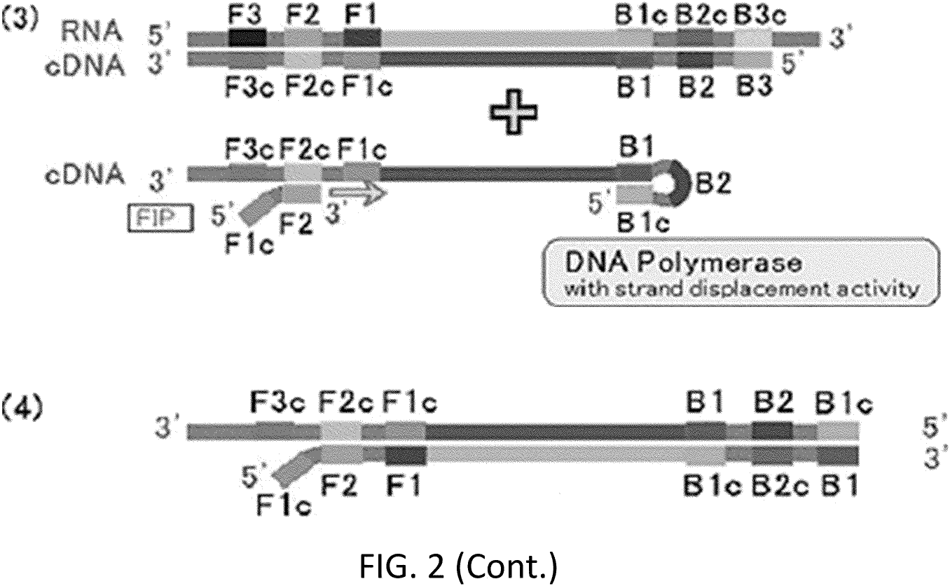

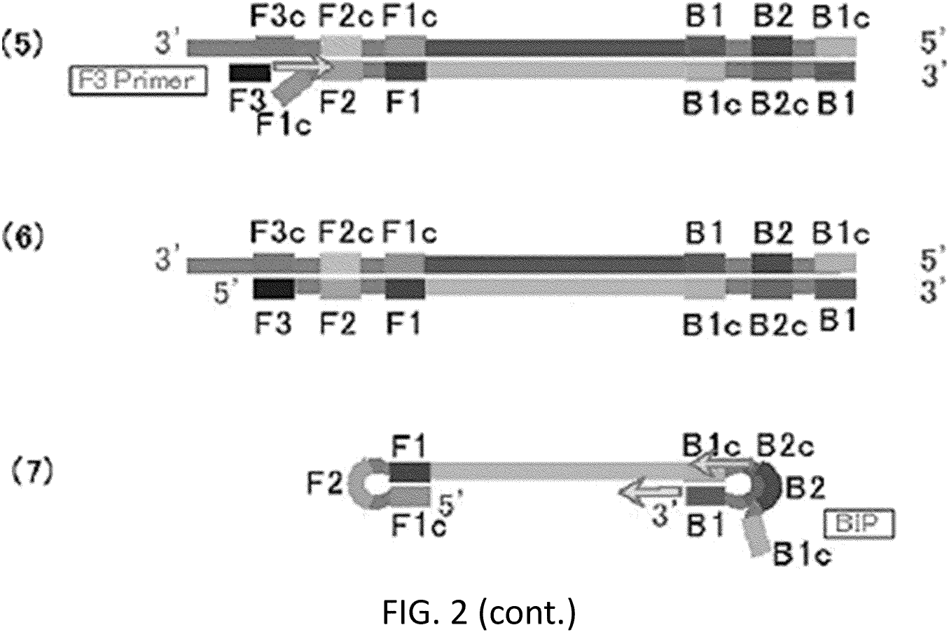

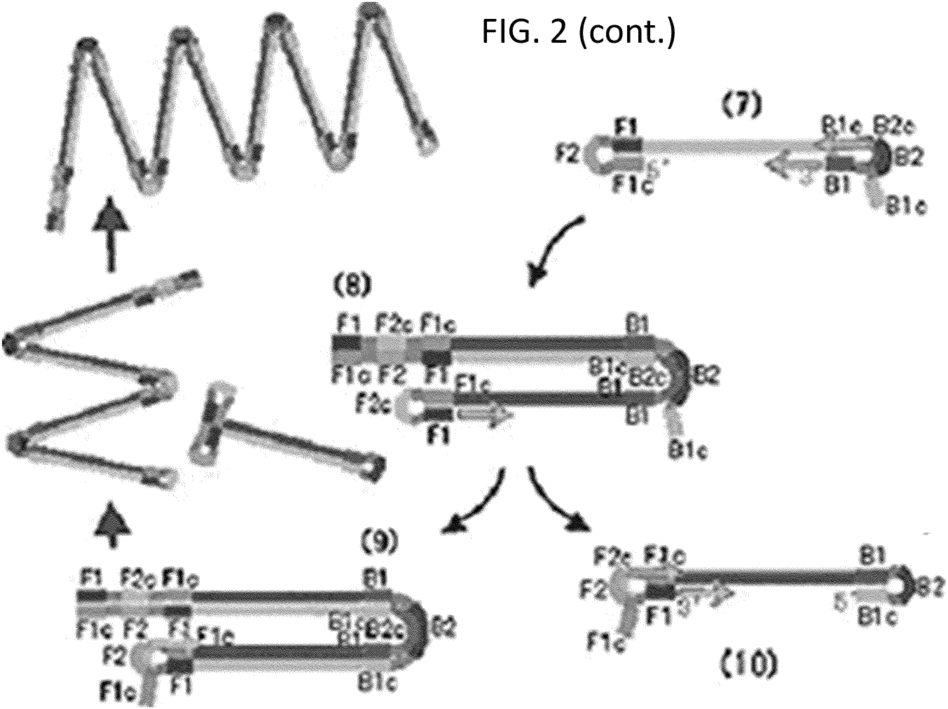

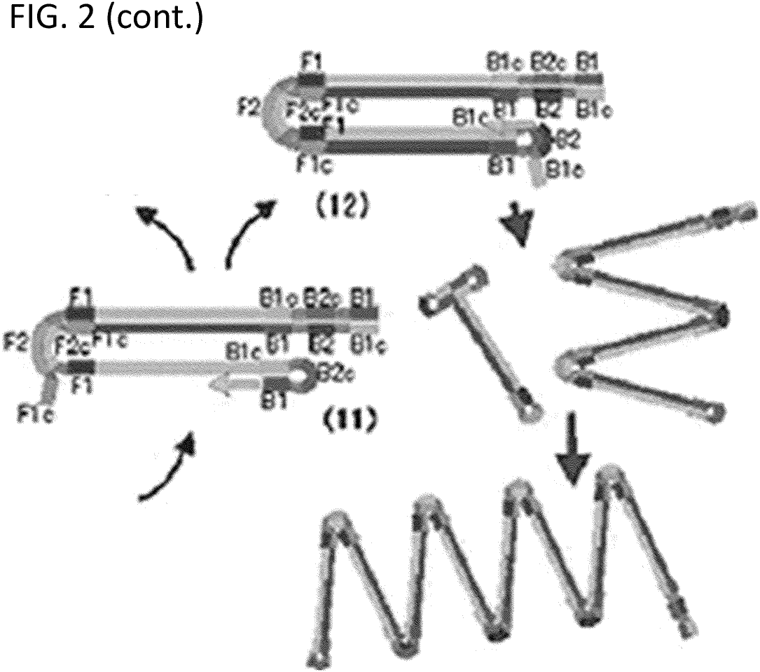

RT-LAMP requires at least four primers that each recognize distinct target sequences within the template strand, including two “internal primers,” i.e., a forward internal primer (FIP) and backward internal primer (BIP) and two “outer primers,” i.e., a forward 3 (F3) primer and a backward 3 (B3) primer. See steps 2-6 of FIG. 2. BIP primers contain two parts: (1) a portion referred to as “B1c” that is identical to a target sequence (also referred to as B1c) that is positioned internally within the template strand, and (2) a portion referred to as “B2” that is complementary to a target sequence (referred to as B2c) that is positioned 3′ to B1c within the template strand. Likewise, FIP primers contain: (1) a portion referred to as “F1c” that is identical to a target sequence (also referred to as F1c) that is positioned internally within the template strand, and (2) a portion referred to as “F2” that is complementary to a target sequence (referred to as F2c) that is positioned 3′ to F1c within the template strand. The outer primers, B3 and F3, anneal to target sequences that are positioned externally relative to the other target sequences within the template strand. Specifically, the B3 primer anneals to a target sequence (referred to as B3c) that is 3′ to B2c in the template strand, and the F3 primer anneals to a target sequence (referred to as F3c) that is 3′ to F2c in the template strand. This design ensures that when DNA synthesis is initiated from the outer primers, it displaces the DNA strands synthesized using the internal primers. Notably, the FIP/F3 primers are designed to anneal to a DNA template that is complementary to the DNA template annealed by the BIP/B3 primers, as each of these sets is used to form one end of a dumbbell-shaped DNA product (described below). Thus, the FIP/F3 primers anneal to the DNA copy produced using the BIP primer, and the BIP/B3 primers anneal to the DNA copy produced using the FIP primer.

The RT-LAMP process can be summarized as follows. First, an internal primer (e.g., BIP) binds to its target sequence in the RNA template and initiates the synthesis of a cDNA strand by reverse transcriptase. This cDNA strand is then displaced from the template strand when synthesis (by DNA polymerase) is initiated from its paired outer primer (e.g., B3). As it is displaced, the single-stranded cDNA product forms a self-hybridizing loop structure due to the inclusion of a reverse complementary sequence in the internal primer sequence (e.g., B1c - B1). This annealing and replacement cycle repeats on the opposite end of the target sequence (initiated by FIP and F3). The resulting product is a short dumbbell-shaped structure that serves as a seed for exponential LAMP amplification.

Optionally, an additional set of “loop primers” can be used to accelerate LAMP amplification. The loop primers, i.e., a loop forward (LF) and a loop backward (LB), are complementary to sequences within the single-stranded loop regions of the dumbbell structure. As amplification proceeds from multiple sites within the dumbbell structure, the amplification products grow and form long concatemers, each with more sites for initiation. This results in rapid accumulation of double-stranded cDNA and amplification byproducts that can be detected by a variety of methods.

FIG. 2 illustrates the RT-LAMP amplification process in greater detail. To perform RT-LAMP, the sample solution is mixed with a reaction solution comprising all the necessary components for this reaction and is incubated at a constant temperature between 60-65° C. In STEP 1, the B2 region of a BIP primer anneals to the B2c sequence within the target RNA, and cDNA is synthesized by reverse transcriptase. In STEP 2, a B3 primer anneals to the B3c sequence within the target RNA, and a new cDNA is synthesized by reverse transcriptase while the cDNA strand formed using the BIP primer is concurrently released. In STEP 3, the F2 region of a FIP primer anneals to the F2c sequence on the opposite end of the released cDNA. In STEP 4, a DNA strand is synthesized off of the FIP primer by a DNA polymerase with strand displacement activity. In STEP 5, a F3 primer anneals the F3c sequence, and a new DNA strand is synthesized while the DNA strand formed using the FIP primer is concurrently released. In STEP 6, the DNA strand synthesized using the F3 primer forms a double-stranded DNA with the DNA strand synthesized using the BIP primer. In STEP 7, since the DNA strand formed using the FIP primer (which was released in step 5) contains complementary sequences at both ends, it self-anneals to form a dumbbell-like DNA structure. This structure becomes the starting structure for LAMP amplification. In STEP 8, the dumbbell-like DNA structure is quickly converted into a stem-loop structure by self-primed DNA synthesis, which unfolds the loop at the 5′ end. The BIP primer anneals to the single-stranded region in the stem-loop DNA to initiate DNA synthesis, which releases the previously synthesized strand. In STEP 9, the released strand forms a stem-loop structure at the 3′ end because it contains complementary F1c and F1 regions. Then, starting from the 3′ end of the F1 region, DNA synthesis initiates using self-structure as a template, and the BIP-linked complementary strand is released. In STEP 10, the released BIP-linked single strand forms the same dumbbell-like structure (7), as its ends contain complementary regions (i.e., F1 - F1c and B1c - B1, respectively). In STEP 11, similar to step 7, the dumbbell-like structure initiates self-primed DNA synthesis starting from the 3′ end of the F1 region. The FIP primer anneals to the F2c region and initiates DNA synthesis. The resulting FIP-linked DNA strand is released by the strand displacement of self-primed DNA synthesis. As in step 10, the same dumbbell-like structure (7) is formed. In STEP 12, a FIP (or BIP) primer anneals to the single stranded F2c (B2c) region of the structure formed in step 9 (or step 12), and DNA synthesis releases double stranded DNA. This amplification method produces structures of various sizes that consist of alternating inverted repeats of the target sequence on the same strand.

### Compositions for Loop-Mediated Isothermal Amplification

In a first aspect, the present invention provides compositions for amplification of a target nucleotide sequence of SARS-CoV-2 by RT-LAMP. The target nucleotide sequence may be any RNA sequence within the SARS-CoV-2 genome. Ideally, the target sequence is “SARS-CoV-2-specific”, meaning that it is not present in the human transcriptome or in genome/transcriptome of other common pathogens. In some embodiments, the target sequence is part of the RNA sequence encoding the Spike protein (S) of the SARS-CoV-2 virus. The S protein is encoded by a 3828-base pair sequence that is located near the 3′ end of the SARS-CoV-2 genome. Advantageously, the S protein is produced from a single open reading frame (ORF) and is not the result of frameshifting, as is the case with other SARS-CoV-2 structural proteins. Additionally, the S protein comprises a furin protease cleavage site (45 base pairs long), which is not found in other coronaviruses such as severe acute respiratory syndrome (SARS) and Middle East respiratory syndrome (MERS). This furin protease cleavage site may be responsible for the increased virulence of SARS-CoV-2 (5).

The S protein may be divided it into three different segments: (1) a 5′ end S target consisting of 1275 bp, (2) an internal S target (comprising the novel furin cleavage site) consisting of 1275 bp, and (3) a 3′ end S target consisting of 1275 bp. One set of the RT-LAMP primers disclosed herein targets the 3′ end S target (i.e., primer set 1), and the other two sets target the internal S target (i.e., primer set 2 and primer set 3).

The present invention provides compositions comprising one or more primers listed in Table 1. The compositions may comprise primer set 1: (i) SEQ ID NO: 1 or SEQ ID NO: 2, (ii) SEQ ID NO: 3, (iii) SEQ ID NO: 10, and (iv) SEQ ID NO: 11; primer set 2: (i) SEQ ID NO: 12, (ii) SEQ ID NO: 13, (iii) SEQ ID NO: 20, and (iv) SEQ ID NO: 21; or primer set 3: (i) SEQ ID NO: 12, (ii) SEQ ID NO: 13, (iii) SEQ ID NO: 71, and (iv) SEQ ID NO: 21.

One exemplary SARS-CoV-2 target sequence is GAUUUUUGUGGAAAGGGCUAUCAUCUUAUGUCCUUCCCUCAGUCAGCACCUCAUG GUGUAGUCUUCUUGCAUGUGACUUAUGUCCCUGCACAAGAAAAGAACUUCACAA CUGCUCCUGCCAUUUGUCAUGAUGGAAAAGCACACUUUCCUCGUGAAGGUGUCUU UGUUUCAAAUGGCACACACUGGUUUG (SEQ ID NO: 69), which corresponds to a portion of the RNA sequence encoding the Spike protein (i.e., positions 573-760). Primer set 1 is effective for amplifying both this target sequence and its DNA analog.

In primer set 1, the FIP primer comprises two subsequences: a F1c sequence comprising AGGGACATAAGTCACATGCAAGAA (SEQ ID NO: 4) and a F2 sequence comprising TTCTTATGTCCTTCCCTCAGT (SEQ ID NO: 5) with a spacer therebetween. Likewise, the BIP primer comprises two subsequences: a B1c sequence comprising AGAAAAGAACTTCACAACTGCTCC (SEQ ID NO: 6) and a B2 sequence comprising CAAAGACACCTTCACGAGG (SEQ ID NO: 7) with a spacer therebetween. The primer set 1 composition may also include loop primers. In some embodiments, it includes one or more loop primers listed in Table 2. Suitably, it comprises the loop primers of SEQ ID NO: 8 and SEQ ID NO: 9.

A second exemplary SARS-CoV-2 target sequence is UUGGUGCAGGUAUAUGCGCUAGUUAUCAGACUCAGACUAAUUCUCCUCGGCGGG CACGUAGUGUAGCUAGUCAAUCCAUCAUUGCCUACACUAUGUCACUUGGUGCAGA AAAUUCAGUUGCUUACUCUAAUAACUCUAUUGCCAUACCCACAAAUUUUACUAU UAGUGUUACCACAGAAAUUCUACCAGUGUCUAUGACCAAGACAUCAGUAGAUUG UACAAUGU (SEQ ID NO: 70), which corresponds to a different portion of the RNA sequence encoding the Spike protein (i.e., positions 1089-1313). Primer set 2 is effective for amplifying both this target sequence and its DNA analog.

In primer set 2, the FIP primer comprises two subsequences: a F1c sequence comprising TAGGCAATGATGGATTGACTAGCTA (SEQ ID NO: 14) and a F2 sequence comprising TTATCAGACTCAGACTAATTCTCC (SEQ ID NO: 15) with a spacer therebetween. Likewise, the BIP primer comprises two subsequences: a B1c sequence comprising AACTCTATTGCCATACCCACAAAT (SEQ ID NO: 16) and a B2 sequence comprising TTGGTCATAGACACTGGTAG (SEQ ID NO: 17) with a spacer therebetween. The primer set 2 composition may also include loop primers. In some embodiments, it includes one or more loop primers listed in Table 2. Suitably, it comprises the loop primers of SEQ ID NO: 18 and SEQ ID NO: 19

The present invention also provides primers that are specifically designed to amplify the Spike protein of the SARS-CoV-2 Delta variant (B.1.617.2), which was first identified in India in late 2020 and spread throughout the world outcompeting pre-existing lineages. The Delta variant is highly contagious and spreads at a much faster rate than the original strain of SARS-CoV-2.

One exemplary SARS-CoV-2 Delta variant target sequence is UUGGUGCAGGUAUAUGCGCUAGUUAUCAGACUCAGACUAAUUCUCGUCGGCGGG CACGUAGUGUAGCUAGUCAAUCCAUCAUUGCCUACACUAUGUCACUUGGUGCAGA AAAUUCAGUUGCUUACUCUAAUAACUCUAUUGCCAUACCCACAAAUUUUACUAU UAGUGUUACCACAGAAAUUCUACCAGUGUCUAUGACCAAGACAUCAGUAGAUUG UACAAUGU (SEQ ID NO: 73), which corresponds to a portion of the RNA sequence encoding the Delta variant Spike (i.e., positions 573-760). Primer set 3 is effective for amplifying both this Delta variant target sequence and its DNA analog.

In primer set 3, the FIP primer comprises two subsequences: a F1c sequence comprising TAGGCAATGATGGATTGACTAGCTA (SEQ ID NO: 14) and a F2 sequence comprising TTATCAGACTCAGACTAATTCTCG (SEQ ID NO: 72) with a spacer therebetween. Likewise, the BIP primer comprises two subsequences: a B1c sequence comprising AACTCTATTGCCATACCCACAAAT (SEQ ID NO: 16) and a B2 sequence comprising TTGGTCATAGACACTGGTAG (SEQ ID NO: 17) with a spacer therebetween. The primer set 3 composition may also include loop primers. In some embodiments, it includes one or more primers included in Table 2. Suitably, it comprises the loop primers of SEQ ID NO: 18 and SEQ ID NO: 19.

TABLE 1

RT-LAMP primers

| Primer set | Name | Label | 5′pos | 3′pos | Length | Tm | Sequence 5′--3′ |
|---|---|---|---|---|---|---|---|
| 1 | Forward 3 | F3 | 573 | 592 | 20 | 61 | GATTTTTGTGGAAAGGGCTATC (SEQ ID NO: 1) or TTTTTGTGGAAAGGGCTATC (SEQ ID NO: 2) |
| | Backward 3 | B3 | 743 | 760 | 18 | 61.2 | CAAACCAGTGTGTGCCAT (SEQ ID NO: 3) |
| | FWD Internal 1-5′ FAM | FIP | | | 48 | 80 | /56-FAM/AGGGACATAAGTCACATGCAAGAATTTTTCTTATGTCCTTCCCTCAGT (SEQ ID NO: 10) |
| | BWD Internal 1 | BIP | | | 47 | 81.5 | AGAAAAGAACTTCACAACTGCTCCTTTTCAAAGACACCTTCACGAGG (SEQ ID NO: 11) |
| 2 | Forward 3 | F3 | 1089 | 1106 | 18 | 56.29 | TTGGTGCAGGTATATGCG (SEQ ID NO: 12) |
| | Backward 3 | B3 | 1291 | 1313 | 23 | 55.79 | ACATTGTACAATCTACTGATGTC (SEQ ID NO: 13) |
| | FWD Internal 1-5′ FAM | FIP | | | 53 | 77.5 | /56-FAM/TAGGCAATGATGGATTGACTAGCTATTTTTTATCAGACTCAGACTAATTCTCC (SEQ ID NO: 20) |
| | BWD Internal 1 | BIP | | | 48 | 78.2 | AACTCTATTGCCATACCCACAAATTTTTTTGGTCATAGACACTGGTAG (SEQ ID NO: 21) |
| 3 | Forward 3 | F3 | 1089 | 1106 | 18 | 56.29 | TTGGTGCAGGTATATGCG (SEQ ID NO: 12) |
| | Backward 3 | B3 | 1291 | 1313 | 23 | 55.79 | ACATTGTACAATCTACTGATGTC (SEQ ID NO: 13) |
| | FWD Internal 1-5′ FAM | FIP | | | 53 | 63.8 | TAGGCAATGATGGATTGACTAGCTATTTTTTATCAGACTCAGACTAATTCTCG (SEQ ID NO: 71) |
| | BWD Internal 1 | BIP | | | 48 | 78.2 | AACTCTATTGCCATACCCACAAATTTTTTTGGTCATAGACACTGGTAG (SEQ ID NO: 21) |

TABLE 2

RT-LAMP loop primers

| | | | | |
|---|---|---|---|---|
| Primer set | Name | Label | Length | Sequence 5′---3′ |
| 1 | 3′Ends2 LF#1 | LF | 20 bp | GACTACACCATGAGGTGCTG (SEQ ID NO: 8) |
| | 3′EndS2 LB#1-5′Biotin | LB | 20 bp | /5Biosg/CAT TTG TCA TGA TGGAAA AG (SEQ ID NO: 9) |
| 2, 3 | Internal S 14-LF | LF | 17 bp | CACTACGTGCCCGCCGA (SEQ ID NO: 18) |
| | InternalS14-LB-5′Biotin | LB | 18 bp | /5Biosg/TTTACTATTAGTGTTACC (SEQ ID NO: 19) |

As used herein, the term “spacer” refers to a nucleic acid sequence that is used to link two polynucleotide sequences. In some embodiments, the spacer is a TTTT linker.

Suitably, one or more of the primers included in the composition contains a region that is at least partially complementary to the target nucleotide sequence of SARS-CoV-2. As used herein, the term “complementary” refers to the ability of a nucleic acid molecule to bind to (i.e., hybridize with) another nucleic acid molecule through the formation of hydrogen bonds between specific nucleotides (i.e., A with T or U and G with C), forming a double-stranded molecule. The phrase “substantially complementary” can refer to at least 85%, 87%, 88%, 89%, 90%, 92%, 94%, 96%, or 98% complementarity.

Suitably, the target nucleotide sequence that is amplified using the compositions disclosed herein is part of the sequence encoding the SARS-CoV-2 spike (S) protein. The primers provided in Table 1 either contain (1) regions that are 100% identical or 100% complementary to the Spike gene of the SARS-CoV-2 2019 variant (i.e., primer set 1 and primer set 2), or (2) regions that are 100% identical or 100% complementary to the Spike gene of the SARS-CoV-2 Delta variant (i.e., primer set 3). The primers show no cross-reactivity with other coronaviruses or other viral and bacterial pathogens. This evaluation was conducted using the Basic Local Alignment Search Tool (BLAST) from the National Center for Biotechnology Information (NCBI).

Suitably, the compositions of the present invention may comprise one or more primers that are labeled to allow for detection of an amplification product prepared by RT-LAMP. The term “label” is used herein to refer to a chemical moiety that is attached (i.e., covalently or non-covalently) to a primer. The label may be used to capture and/or detect the RT-LAMP amplification products. Exemplary detectable labels include, for example, a fluorescent label, a chemiluminescent label, a quencher, a radioactive label, an antibody, biotin, and gold. For example, as shown in Table 1 and Table 2, the forward internal primers (FIP) and backward loop primers (BIP) may contain 5′ labels (e.g., FAM or biotin), such that the resulting amplification products can be detected by lateral flow assay (LFA) (e.g., using anti-FAM antibody or streptavidin-labeled latex beads). In an alternative embodiment, the backward internal primer (BIP) and loop forward (LF) primers may contain 5′ labels.

In some embodiments, the composition comprises one or more additional reagents for use in RT-LAMP. Exemplary RT-LAMP reagents include, without limitation, deoxynucleotide triphosphates (dNTPs), a DNA polymerase, a reverse transcriptase, buffering agents, primers for detecting a nucleotide control sequence, enzyme co-factors, positive control nucleic acids, detergents, inactivation buffers, dilution buffers, a viral transport medium (VTM), or dye.

As used herein, an “inactivation buffer” is a buffer than inactivates RNases in a sample. Suitable inactivation buffers include those that comprise an effective amount of one or more of TCEP, guanidium hydrochloride, and EDTA.

As used herein, a “dilution buffer” is a buffer used to add more solvent to a given amount of solute. Suitable dilution buffers for use with the present invention include buffers that comprise an effective amount of a buffering salt (e.g., citrate) to buffer the diluted sample to a pH within the range of 5-8.

A “viral transport medium” (VTM) is a solution used to preserve virus specimens after collection so that they can be transported and analyzed in a laboratory at a later time. Virus samples are prone to degradation unless they are stored in an ultra-low temperature freezer or in liquid nitrogen. However, such cooling equipment is seldom available in the field. Thus, a VTM is used to store virus specimens at ambient temperatures. Suitable VTM components include, without limitation, saline solution, phosphate-buffered saline (PBS), or fetal bovine serum (FBS). In some embodiments, the VTM is Universal Transport Medium™ (UTM®). UTM® is an FDA cleared collection and transport system suitable for collection, transport, preservation, and long-term freeze storage of clinical specimens containing viruses for viral molecular diagnostic testing, including SARS-CoV-2. The transport medium comes in a plastic, screw cap tube and maintains organism viability for 48 hours at room or refrigerated temperature.

In some embodiments, the composition additionally includes components for loop-mediated isothermal amplification of a “control nucleotide sequence” from the subject, i.e., a nucleotide sequence that is found in the genome of the subject but not in the genome of SARS-CoV-2. Detection of such sequences can be used to verify that the assay is working. Thus, in embodiments in which the subject is a human, the compositions may also include primers and reagents for RT-LAMP amplification of one or more human genes for use as a control nucleotide sequence. Exemplary human genes that can be used as a control nucleotide sequence include, without limitation, human ribonuclease P30 (RRP30), human albumin (HA), human actin beta (HAB), human actin gamma (HAG), human tubulin alpha (HTB), human saliva amylase variant #1 and variant #2 (AMY1AV1 and AMY1AV2), ribonuclease P (RNAseP), apolipoprotein L1 (ApoL1), and glyceraldehyde 3-phosphate dehydrogenase (GAPDH). Exemplary primer sets for the amplification of these control genes are provided in Tables 3-9.

TABLE 3

Primers for amplifying human albumin, set 1

| Name | Label | 5′pos | 3′pos | Length | Sequence 5′ - 3′ |
|---|---|---|---|---|---|
| Forward3 S1 | F3 | 496 | 517 | 22 | GTTCAGATTCTAAAACAGTGCT (SEQ ID NO: 22) |
| Backward3 S1 | B3 | 692 | 711 | 20 | TGTCCAATACAGTAGCCATT (SEQ ID NO: 23) |
| FWD Internal S1 | FIP | | | 43 | AGTGACTACCTTATTGGATAGCCCTTTTCTCGTAGAGTTTTCTGCGT (SEQ ID NO: 24) |
| BWD Internal S1 | BIP | | | 47 | TATGACAAGTGCAACTGAGAAACATTTTGCCACATGTGACTATATACATTC (SEQ ID NO: 25) |
| Albumin LF#1 | LF | | | 21 | AGATACAGAATATCTTCCT CA (SEQ ID NO: 26) |
| Albumin LB#1 | LB | | | 20 | GAAACGAGGGGTTACTAATC (SEQ ID NO: 27) |
| Albumin LB#1-Biotin | LB #1-Biotin | | | 20 | /5Biosg/GAAACGAGGGGTTACTAATC (SEQ ID NO: 27) |
| Albumin FIPS1-FAM | FIPS1-FAM | | | 47 | /56-FAM/AGTGACTACCTTATTGGATAGCCCTTTTCTCGTAGAGTTTTCTGCGT (SEQ ID NO: 24) |

TABLE 4

Primers for amplifying human albumin, set 2

| Name | label | 5′pos | 3′pos | Length | Sequence 5′-----3′ |
|---|---|---|---|---|---|
| Forward3 S2 | F3 | 1340 | 1360 | 21 | ACAAAAAAATTAGGCATGGTG (SEQ ID NO: 28) |
| Backward3 S2 | B3 | 1504 | 1522 | 19 | AAACCCCACAGATTTCCTT (SEQ ID NO: 29) |
| FWD Internal S2 | FIP | | | 41 | CTCTCAGGTCCAAACGATCCTCTTTTCACATGCCTGTAGTCCTAG (SEQ ID NO: 30) |
| BWD Internal S2 | BIP | | | 42 | ACAAAAAAATTAGGCATGGTG (SEQ ID NO: 31) |

TABLE 5

Primers for amplifying human albumin, set 3

| Name | Label | 5′pos | 3′pos | Length | Sequence 5′-----3′ |
|---|---|---|---|---|---|
| Forward3 S3 | F3 | 1586 | 1605 | 20 | GGCATACAATTTGCTTGCTT (SEQ ID NO: 32) |
| Backward3 S3 | B3 | 1768 | 1789 | 22 | ATCATGTATGGGTATAGGTTCT (SEQ ID NO: 33) |
| FWD Internal S3 | FIP | | | 47 | CGCAAAGAGAATGGTAGTTCCCTAATTTTGTGTGTGCATAGATCTACTGAC (SEQ ID NO: 34) |
| BWD Internal S3 | BIP | | | 46 | GGAAGCCACATATGCCTATCTAGGTTTTTCTTCTTCACCATTATCCAGAA (SEQ ID NO: 35) |

TABLE 6

Primers for amplifying human albumin, set 4

| Name | Label | 5′pos | 3′pos | Length | Sequence 5′-----3′ |
|---|---|---|---|---|---|
| Forward3 S4 | F3 | 1602 | 1621 | 20 | GCTTAATCTATGTGTGTGCA (SEQ ID NO: 36) |
| Backward3 S4 | B3 | 1768 | 1789 | 22 | ATCATGTATGGGTATAGGTTCT (SEQ ID NO: 37) |
| FWD Internal S4 | FIP | | | 42 | GCTTCCTACGCAAAGAGAATGGTATTTTAGATCTACTGACACACGC (SEQ ID NO: 38) |
| BWD Internal S4 | BIP | | | 47 | ATATGCCTATCTAGGCCTCAGATCTTTTATCTTCTTCACCATTATCCAGAA (SEQ ID NO: 39) |

TABLE 7

Primers for amplifying human albumin, set 5

| Name | Label | 5′pos | 3′pos | Length | Sequence 5′-----3′ |
|---|---|---|---|---|---|
| Forward3 S5 | F3 | 1626 | 1644 | 19 | TCTACTGACACACGCATAC (SEQID NO: 40) |
| Backward3 S5 | B3 | 1796 | 1813 | 18 | ACAGGTTGCTACGCTAGA (SEQ ID NO: 41) |
| FWD Internal S5 | FIP | | | 48 | TATGATCTGAGGCCTAGATAGGCATTTTATTAGGGAACTACCATTCTCTTTG (SEQ ID NO: 42) |
| BWD Internal S5 | BIP | | | 47 | GAATAGGCTTTCTGGATAATGGTGATTTTATCATGTATGGGTATAGGTTCT (SEQ ID NO: 43) |

TABLE 8

Primers for amplifying AMY1A variant 1

| Name | Label | 5′pos | 3′pos | Length | Sequence 5′-----3′ |
|---|---|---|---|---|---|
| Forwad3 S1 | F3 | 81 | 98 | 18 | ATCTAGAGGCTGGGAAGG (SEQ ID NO: 44) |
| Backward3 S1 | B3 | 260 | 278 | 19 | AGTGTCCTTTCCAGAAACT (SEQ ID NO: 45) |
| FWD Internal S1 | FIP | | | 40 | CCTATGGCTTGGACTTTCCAACTTTTGCTCCTGAACCAGTTGTT (SEQ ID NO: 46) |
| BWD Internal S1 | BIP | | | 42 | AGCTTACGTTATCTACCAGAGCATTTTGGTATAAATGCGAACCGCT (SEQ ID NO: 47) |

TABLE 9

Primers for amplifying human actin beta

| Name | Label | 5′pos | 3′pos | Length | Sequence 5′-----3′ |
|---|---|---|---|---|---|
| Forward3 S2 | F3 | 546 | 565 | 20 | GCAGACATACAACGGACGGT (SEQ ID NO: 48) |
| Backward3 S2 | B3 | 734 | 749 | 16 | ACCTACCCAGCCGCAG (SEQ ID NO: 49) |
| FWD Internal S2 | FIP | | | 41 | CGTTAGTGGGTGACCTAGGCACTTTTCCAGACCCAGGCTGTGTAG (SEQ ID NO: 50) |
| BWD Internal S2 | BIP | | | 39 | ACCCTCAAAAGCAGGCAGCTTTTTGGAAGGAAGGTGGGCTCTA (SEQ ID NO: 51) |

Use of the disclosed compositions results in the generation of a dumbbell-shaped nucleic acid that forms the seed for exponential LAMP amplification. This nucleic acid comprises in order from 5′ to 3′ : F1c, F2, a nucleotide sequence substantially complementary to F1c (i. e., F1), B1c, B2, and a nucleotide sequence substantially complementary to B1c (i.e., B1). F1c and F1 hybridize and B1c and B1 hybridize, such that the nucleic acid forms a dumbbell structure. This dumbbell structure contains multiple sites for the initiation of DNA synthesis. As amplification proceeds from these multiple initiation sites, the products grow and form long catcatemers, (i.e., amplification products) that can be detected by a variety of methods. See Steps 8-12 above. Thus, these dumbbell-shaped nucleic acids and amplification products thereof are also provided by the present invention.

### Methods

The present invention also provides methods for detecting SARS-CoV-2 in a sample. The methods comprise (a) contacting the sample with a composition described herein under conditions sufficient for loop-mediated isothermal amplification of the target nucleotide sequence of SARS-CoV-2, and (b) detecting the presence or absence of an amplification product of the target nucleotide sequence. Conditions sufficient for amplification of the target nucleotide sequence may comprise an effective temperature of from 55-70° C. and/or an effective amplification time of between 20-60 minutes.

The sample may be obtained from a subject having or suspected of having a SARS-CoV-2 infection. Any sample that would potentially contain SARS-CoV-2 virus can be utilized. In some embodiments, the sample is obtained from the upper or lower respiratory tract of the subject. Exemplary samples include, without limitation, saliva samples, bronchoalveolar lavages, sputum samples, nasopharyngeal samples, nasal samples, oropharyngeal samples, sewage, and stool samples. In preferred embodiments, the sample is a nasopharyngeal swab, oropharyngeal (throat) swab, mid-turbinate nasal swab, anterior nasal swab, nasopharyngeal wash/aspirate, or nasal aspirate

As used herein, the terms “subject” and “patient” refer to both mammals and non-mammals. A “mammal” may be any member of the class Mammalia including, but not limited to, humans, non-human primates (e.g., chimpanzees, other apes, and monkey species), farm animals (e.g., cattle, horses, sheep, goats, and swine), domestic animals (e.g., rabbits, dogs, and cats), or laboratory animals including rodents (e.g., rats, mice, and guinea pigs). Examples of non-mammals include, but are not limited to, birds, and the like. The term “subject” does not denote a particular age or sex. In preferred embodiments, the subject is a human. In some embodiments, the subject has or is suspected of having a SARS-CoV-2 infection.

Performing RT-LAMP amplification of SARS-CoV-2 RNA using labeled primers generates labeled amplicons (e.g., amplicons labelled with FITC and biotin that can be detected by lateral flow assay (LFA) using anti-FITC antibody and streptavidin-labeled latex beads). Exemplary methods and devices for detecting the labeled amplicons include those described in U.S. Pat. Pub. No. 2009/0181388 and 2011/0229887. In some embodiments, the amplicons are detected visually without instrumentation. In some embodiments, the amplicons are detected using dyes that recognize by-products of DNA synthesis, such as intercalating dyes. Examples of suitable dyes include, without limitation, malachite green, calcein, hydroxynaphthol blue, and pH-sensitive dyes. Exemplary dyes and methods are disclosed in Nzelu et al. Acta Trop, 132, 1-6 (2014); Tomita et al. Nat Protoc, 3, 877-882. (2008); Goto et al. Biotechniques, 46, 167-172 (2009); and Tanner et al, Biotechniques, 58, 59-68 (2015). In some embodiments, the amplicons are detected using fluorescent molecular beacon probes. In some embodiments, the amplicons are detected electrochemically. These and other methods for detecting amplicons are described in Taylor J. Moehling, et al. Expert Review of Molecular Diagnostics, 21:1, 43-61 (2021).

In some embodiments, the methods further comprise extracting RNA from the sample prior to the contacting step. The RNA may be extracted by lysing the SARS-CoV-2 virus present in the sample. The terms “lysis” or “lyse” are used to describe the disruption of cells or viral particles within a sample to gain access to materials within. Methods of lysis include, but are not limited to, chemical lysis, thermal lysis, mechanical lysis, and osmotic lysis. The term “lysate” is used to refer to the sample following a lysis procedure. In some embodiments, the RNA is extracted using a heat lysis method. Heat lysis can be performed by heating the sample to an effective temperature for an effective amount of time to disrupt the viral envelope and release the viral genomic RNA. In other embodiments, the RNA is extracted using a chemical lysis method. Chemical lysis can be performed using reagents such as detergents and/or chaotropic salts.

In some embodiments, the RNA in the lysate is protected from ribonucleases (RNases) via treatment with an RNase inactivation buffer. Suitable inactivation buffers include reducing agents such as TCEP (tris(2-carboxyethyl)phosphine), chaotropic agents such as guanidium hydrochloride, and chelating agents such as EDTA (ethylenediaminetetraacetic acid). The lysed RNA may be further stabilized using a buffering salt such as sodium citrate. To this end, in some embodiments, the sample is treated with an RNase inactivation buffer prior to the contacting step. In some embodiments, the methods further comprise isolating or purifying the extracted RNA. The RNA may be purified using standard methods that are well known in the art, including those that rely on organic extraction, ethanol precipitation, silica-binding chemistry, cellulose-binding chemistry, and ion exchange chemistry. Many reagents and kits for RNA extraction are commercially available. In some embodiments, the RNA is isolated or purified using microspheres, as described in International Patent Pub. No WO/2019/183263.

The methods described herein may also include the amplification of a control nucleotide sequence that originates from the subject, and detection of the presence of an amplification product generated from the control sequence. As is described above, the control sequence may comprise part of a human gene.

### Diagnostic Devices

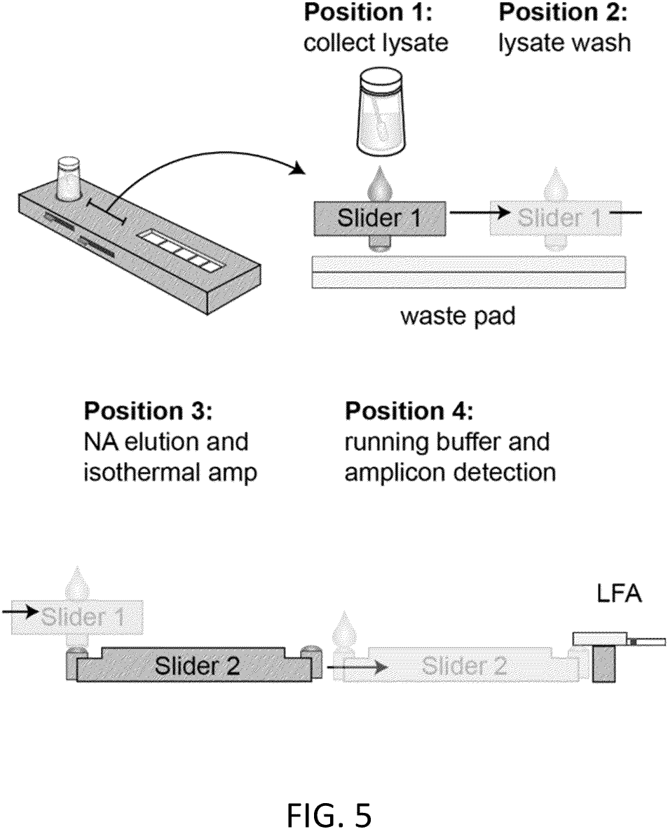

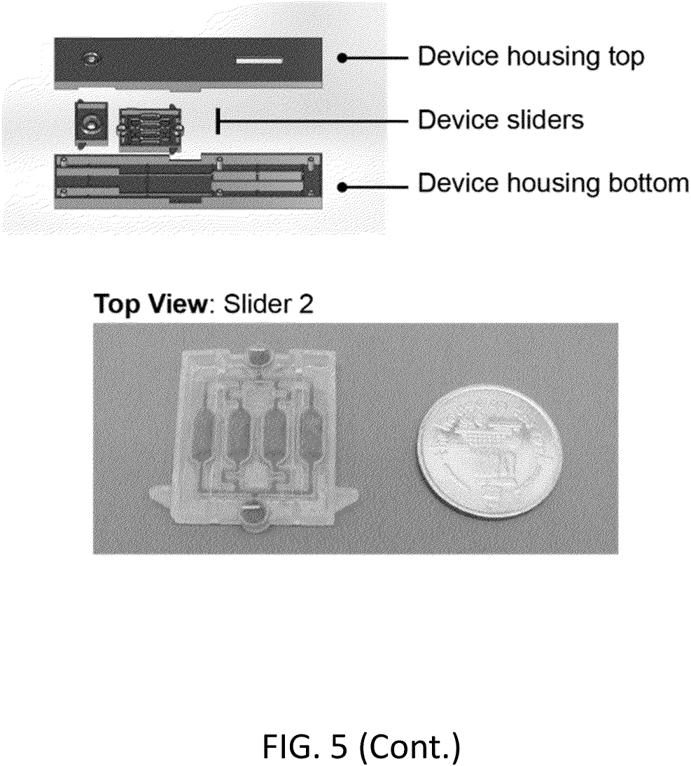

The present invention also provides diagnostic devices. In some embodiments, the device comprises a housing with a sample inlet for receiving the sample. The housing contains the following within it: (a) a moveable nucleic acid binding stage comprising a permeable nucleic acid binding substrate and an eluate outlet by which an eluate may exit the binding stage; (b) a wash reservoir containing a wash buffer; (c) an elution reservoir containing an elution buffer; (d) a moveable amplification stage comprising an eluate inlet for receiving the eluate from the nucleic acid binding stage, at least one reaction chamber comprising a nucleic acid amplification reagent, and an amplicon outlet by which an amplified sample may exit the amplification stage; (e) a running buffer reservoir containing a running buffer; and (f) a detection device that provides a readout that indicates whether the target nucleic acid is present in the sample. Importantly, the nucleic acid binding stage is configured such that it can be positioned to be in fluid communication with the sample inlet, the wash reservoir, the elution reservoir, and the amplification stage via the eluate outlet. Additionally, the amplification stage is configured such that it can be positioned to be in fluid communication with the running buffer reservoir and the detection device via the amplicon outlet. See FIG. 5 for a schematic illustration of one exemplary device.

As used herein, the term “housing” refers to a rigid casing that encloses and protects the device components. The housing may comprise a sample inlet for receiving a sample or sample lysate. Advantageously, the devices of the present invention are self-contained, limiting the potential for exposure to any harmful or infectious materials found in the sample. The housing also prevents contamination of the testing environment with the highly amplified DNA products from RT-LAMP, which can lead to false positives. Suitably, the housing may comprise one or more readout windows that allow a user to inspect a readout of a detection device within the housing thereby avoiding the need to open the housing and potentially expose the user to harmful or infectious materials.

The nucleic acid binding stage is initially positioned directly beneath the sample inlet to receive the sample and direct it into the permeable nucleic acid binding substrate. This substrate binds nucleic acids and filters out large particulate as the sample seeps through it. Advantageously, the nucleic acid binding substrate can be positioned at the bottom of a funnel built into the nucleic acid binding stage, such that the sample is directed into the substrate. The nucleic acid binding substrate may comprise any permeable material that can bind nucleic acids in a complex matrix and can release the nucleic acids when contacted with an eluent. Advantageously, the substrate binds nucleic acids in a nonspecific manner, allowing it to be used to separate any nucleic acid from a sample without having to be tailored for a specific nucleic acid target. Suitable nucleic acid binding substrates include, without limitation, cellulose membranes, cellulose-coated beads, chitosan membranes, chitosan-coated beads, silica columns, aluminum oxide membranes, and the like. In some embodiments, the nucleic acid binding substrate is a silica membrane and/or silica microspheres. For example, borosilicate glass microspheres may be used for DNA extraction as disclosed in International Patent Pub. No. WO/2019/109092, which is incorporated by reference herein.

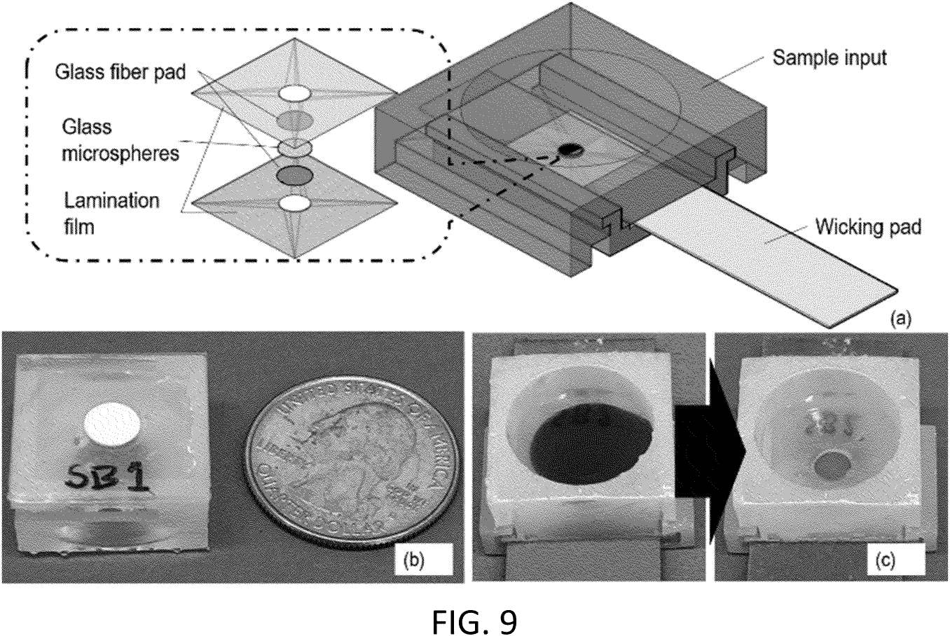

In some embodiments, the binding substrate comprises silica microspheres and fiber pads (e.g., paper, silica, cellulose, etc.) or membranes. This substrate combines the advantages of silica-bead-based and paper-based nucleic acid extractions while eliminating their drawbacks. To form this substrate, micrometer-sized silica beads are packed between two pieces of fiber pads via a low-cost, readily deployable lamination process (FIG. 9). The laminated beads will bind nucleic acids and allow the use of a paper, or a similar material, with larger pore sizes for fast flow and clogging prevention. The use of fiber pads and lamination not only simplifies the silica beads packing process but also enables the use of capillary forces for liquid transfer. The binding of nucleic acids is achieved using a combination of chaotropic agents, such as guanidium isothiocyanate and ethanol, and a co-precipitant such as glycogen and glycoblue™. Exemplary nucleic-acid-capture microspheres and their production and use are described in International Patent Pub. No. WO/2019/109092.

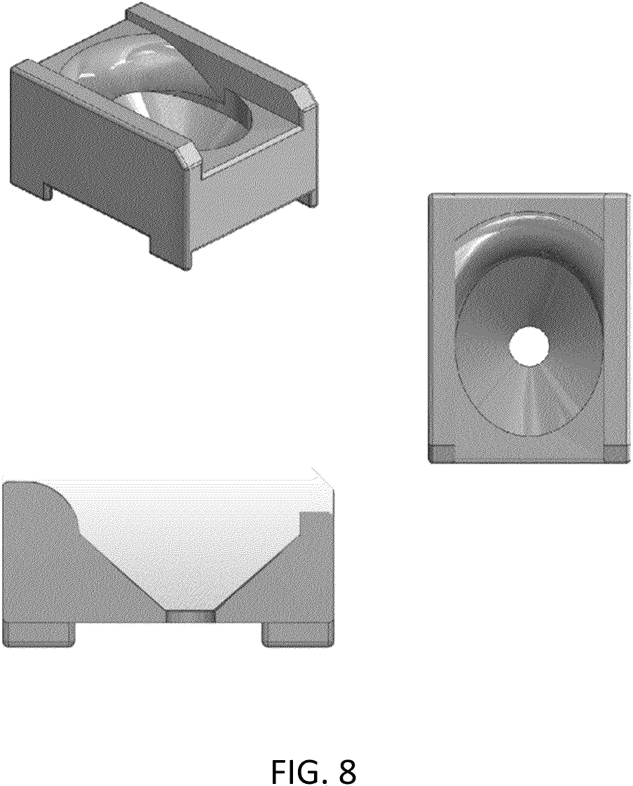

The binding stage may comprise a collection funnel, binding membrane, and sponge ramp (FIG. 8). The funnel collects liquids as they are introduced to the binding stage. First in the liquid collection sequence, the lysate drains from the lysis cartridge, through the inlet of the processing/detection device and into the collection funnel. The funnel channels the lysate through the binding substrate, which collects nucleic acids as the lysate passes through. The devices are designed to be operated by simple, limited, manual interventions, such as sliding a movable component or pressing down a collapsible chamber. In some embodiments, the device may comprise two moveable stages: the nucleic acid binding stage and the amplification stage. In some embodiments, the nucleic acid binding stage is configured to slide between positions of fluid communication with the sample inlet, the wash reservoir, and the elution reservoir, and the amplification stage is configured to slide between positions of fluid communication with the elution reservoir, the running buffer reservoir, and the detection device.

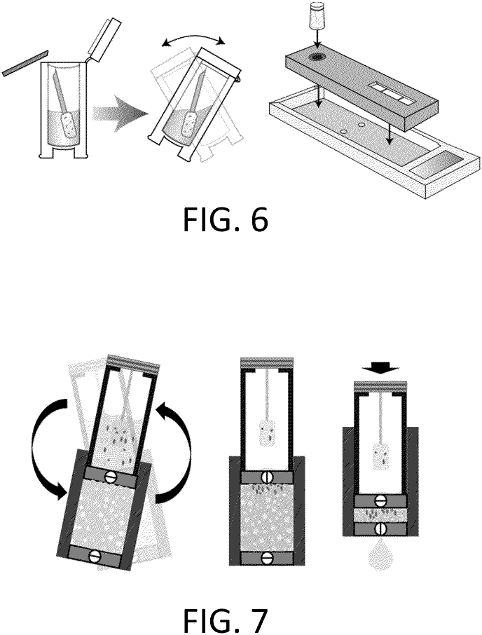

In some embodiments, the device is intended to be used with a reusable, handheld controller that moves internal components within the processing device in an automated fashion (FIG. 6). The reusable controller removes user manual steps previously required, simplifying platform operation, which increases success rate by removing variability. The handheld platform is portable so it can be used throughout a single clinic or in mobile field clinics, among other unique diagnostic settings.

The binding stage or amplification stage may be controlled using a handheld controller and moved using a force, such as mechanical or electromagnetic force. Stage movements may be automated by a single-board microcontroller that is coded to control a small electric motor, such as a stepper motor. The small electric motor may spin a lead screw that is threaded with a moving stage. Features attached to this moving stage, such as a magnet or clips or similar physical features, connect with the binding and amplification stage within the processing/detection device. The program installed onto the microcontroller moves the electric motor at specific speeds at specific times, according to the protocol designed to collect, amplify, and detect target nucleic acid sequences.

The microcontroller, electric motor, and external force application stage are all housed within a reusable device or controller. The controller will be made of polymer material that can be easily decontaminated with standard wipes or spray, preventing cross-contamination and the risk of passing infections from one person to another. The reusable controller is designed to be portable so one can be used throughout a doctor’s office or in a mobile clinic setting. The reusable controller has a user interface and buttons that allows the user to select a protocol specific to a sample or target type.

The heating element that is required for nucleic acid amplification will be powered by the reusable controller. The battery pack that powers the microcontroller and the electric motor also powers the heating element. The microcontroller will command the temperature and reaction time. The heating element could be housed within the controller or within the processing/detection device and connected to the power source via external nodes.

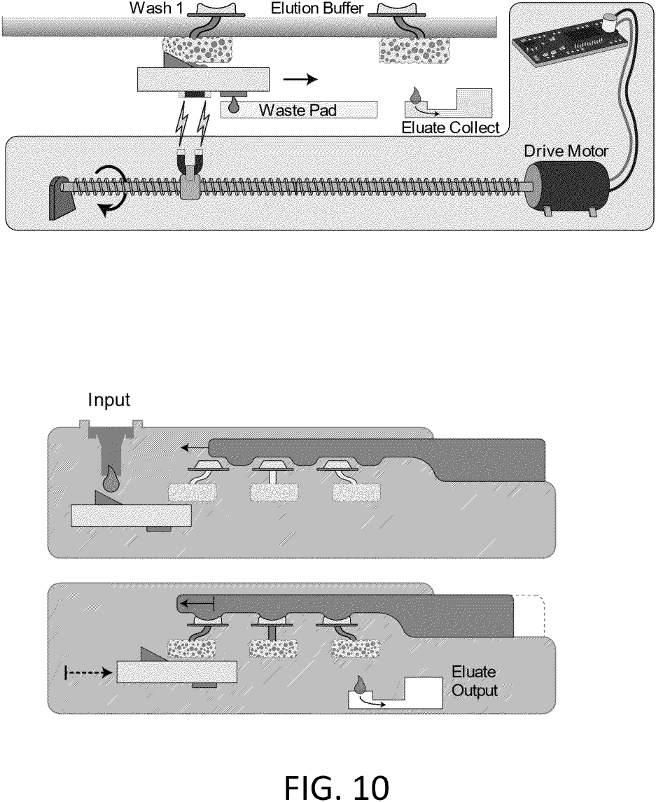

Additionally, the device may comprise one or more reservoirs. In some embodiments, the device comprises four buffer reservoirs: the wash reservoir, elution reservoir, rehydration buffer reservoir, and running buffer reservoir. A reservoir is suitably liquid-tight and/or airtight to allow the contents to be maintained for extended periods of time in ambient conditions until needed for use. In some embodiments, one of more of these reservoirs comprise a blister pouch (e.g., an aluminum foil blister pouch). Suitably the reservoir’s contents can be accessed when the appropriate stage is positioned to be in fluid communication with the reservoir. For example, the stage may be configured to puncture the blister pouch when the stage is in position to receive the reservoir’s contents. Prepackaged buffers can be strategically incorporated along the sliding path of each stage. In this configuration, the operator slides the stages against the buffer reservoirs, puncturing the pouches to deliver the proper volume of each buffer in sequence (FIG. 5). In another embodiment, reservoirs are pierced and drained into sponges for temporary storage as they await their turn in the protocol sequence. The sponge ramp gently compresses the sponges, draining reagents into the funnel of the binding stage (FIG. 10). The sponge ramp of the binding stage may be built up from the collection funnel. The purpose of the ramp is to compress reagent sponges as the binding stage moves along the protocol track. The reagent sponges are a passive way to temporarily store reagents after they are expelled from their shelf-life storage reservoirs. To optimize the work done by the limited number of user steps required to use the device, some or all of the shelf-life reagent reservoirs are burst all at once. The reservoirs are pierced and drained into the reagent sponges through mechanical actuation as the processing/detection device is inserted into a reusable controller. From there, the sponges saturate with their respective reagent, holding it until compressed by the sponge ramp. As the ramp moves beneath a sponge that is saturated with a specific buffer, it gently compresses the sponge, draining it of a set volume of liquid into the stage. The drained reagents flow into the collection funnel via gravity and are wicked through the binding membrane via fluidic forces.

The wash reservoir may contain any suitable wash buffer for nucleic acid extraction. As used herein, a “wash buffer” is a substance capable of removing impurities adsorbed onto the surface of the nucleic acid binding substrate. The wash buffer should be selected such that nucleic acids adsorbed onto the nucleic acid binding substrate are not extracted when the wash buffer contacts the substrate. Suitably, the wash buffer may be selected from water, an alcohol (e.g., ethanol or isopropanol), medium salt buffer (e.g., 100 mM or 200 mM NaCl), or combinations thereof. In some embodiments, the wash buffer is ethanol diluted to a concentration of 30% or less by volume.

The elution reservoir may contain any suitable elution buffer that is able to separate nucleic acid material from the nucleic acid binding substrate. The elution solution is preferably an aqueous solution of low ionic strength. In some embodiments, the elution buffer is water or TE buffer (i.e., Tris-HCl ethylenediamine-tetraacetic acid (EDTA)). However, other elution buffers suitable for use with this invention will be readily apparent to one skilled in this art.

The running buffer reservoir may contain any running buffer that is compatible with the detection device. Suitable running buffers include, without limitation, water, phosphate buffered saline (PBS), saline-sodium citrate (SSC) buffer, Tris buffer, MES buffer, HEPES buffer, or any other buffer suitable for buffing at a pH between 7.4 and 8.0. In some embodiments, the running buffer is a buffered salt solution containing a detergent, such as 75mM Sodium Borate, pH 7.4, 0.25% Tween 20.

In some embodiments, the device includes a waste pad positioned beneath the nucleic acid binding stage to absorb liquid (i.e., sample and buffer) that passes through the nucleic acid binding substrate. The waste pad may take any form and may comprise any absorptive material, provided that it does not disrupt device function.

The devices comprise at least one reaction chamber in which a nucleic acid amplification reaction may be performed. The reaction chamber contains a nucleic acid amplification reagent. In some embodiments, this reagent comprises the basic factors required for DNA amplification, i.e., a DNA polymerase, one or more primers, and nucleoside triphosphates. In some embodiments, the nucleic acid amplification reagent is lyophilized and is reconstituted as the eluate flows into the reaction chamber. Use of a lyophilized reagent is advantageous for a point-of-care device because such reagents remain stable for a long time at room temperature.

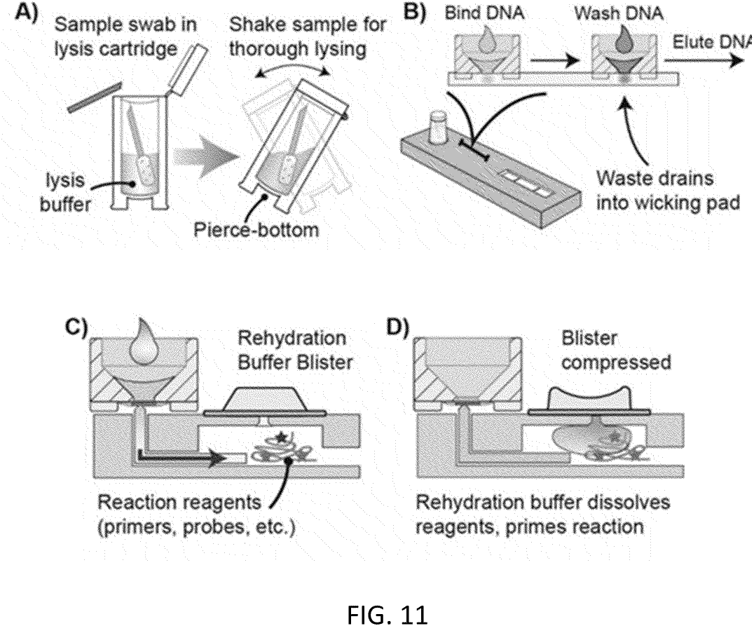

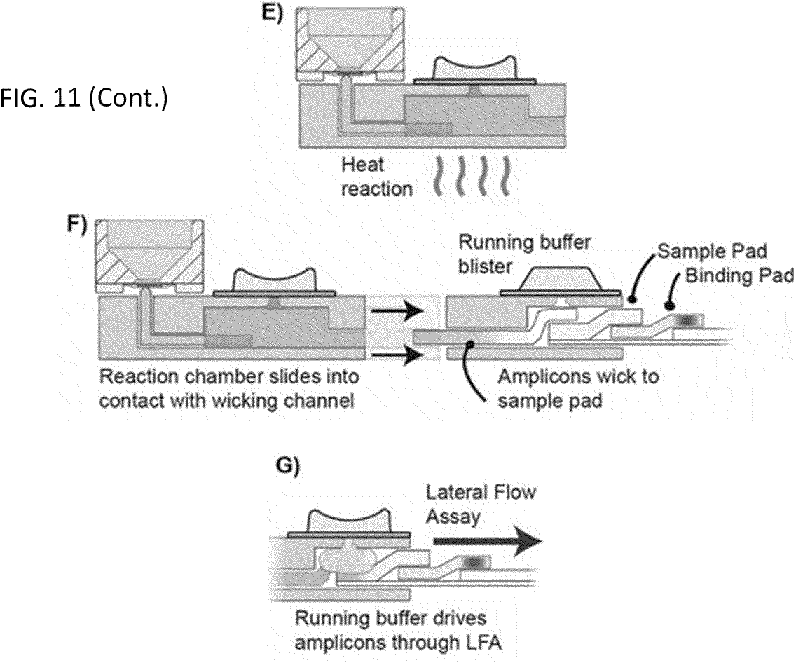

After the extracted nucleic acids have been washed, the outlet of the binding stage is positioned above the inlet of the reaction stage. When this occurs, a fluidic connection is made between the two stages, draining eluate from the binding substrate into a capillary microchannel. The microchannel wicks eluate via capillary action into one or several reaction chambers. This wicking could be done through any sort of hydrophilic microchannel. One such example is a paper microchannel that comes into contact with the bottom of the binding membrane (FIG. 11)

The reaction chambers may be pre-packaged with lyophilized reaction reagents, including primers, probes, and magnesium. At this point the rehydration buffer reservoir is pierced, draining into the reaction chamber and mixing all reagents. Once the rehydration reservoir is burst, the heating element begins warming the reaction to the desired temperature for the desired duration. The reaction temperature and duration are specific for the desired nucleic acid target that is being amplified.

Once the reaction is complete, the reaction chamber is brought into fluidic contact with a capillary microchannel, similar to the previous microchannel (FIG. 11). This downstream microchannel wicks the reaction products (i.e., amplicons) towards the sample pad of the lateral flow assay used for target detection. After the amplicons have been wicked into the sample pad, the running buffer reservoir is pierced. As the running buffer drains into the sample pad, it drives the amplicons to wick through the binding pad and eventually through the test strip of the lateral flow assay. The lateral flow assay then works as any standard lateral flow diagnostic test. The user can read the test lines that arise over the next few minutes and determine results. The device could also be read by an automated reading device that is built into the reusable controller.

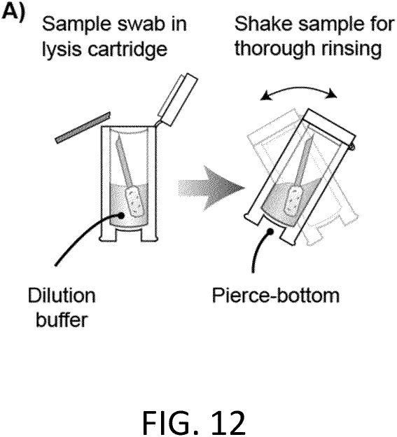

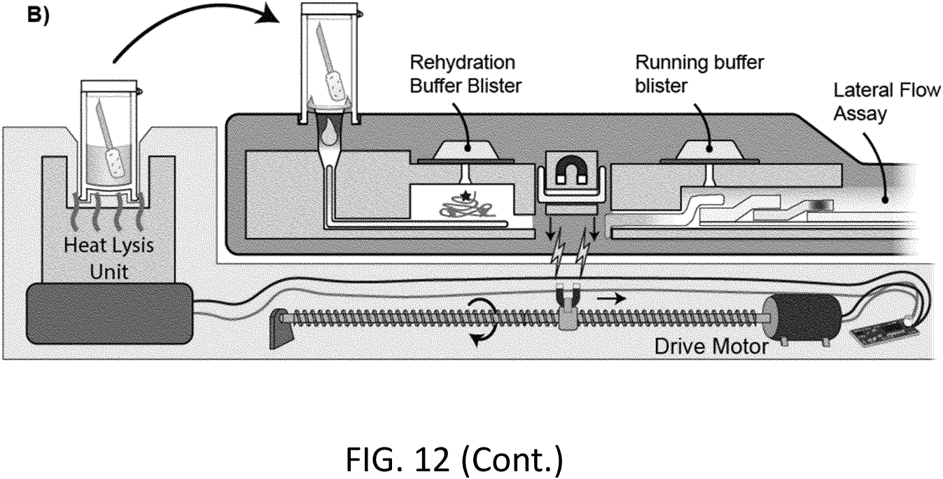

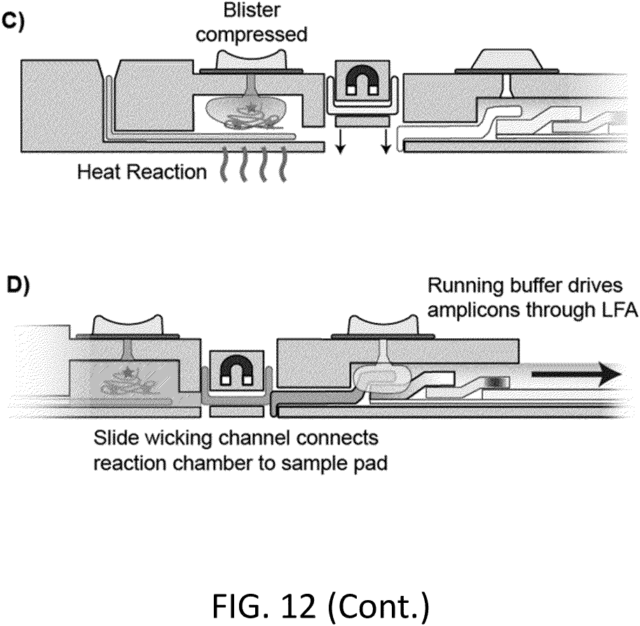

Another exemplary embodiment of the invention is illustrated in FIG. 12. This embodiment takes advantage of “direct-to-amplification” assay techniques and does not require nucleic acid extraction, wash, or elution, thus, reducing the number of automated steps. This platform is best suited for samples that are more conducive to direct-to-amplification techniques, such as nasal swabs, nasopharyngeal swabs, oropharyngeal swabs, and saliva (FIG. 12A). Here, the sample will be heat lysed within the lysis cartridge in a heating slot built into the reusable controller (FIG. 12B). Following lysis, the tube is transferred to the sample inlet of the diagnostic device. Note how, in this design, the sample inlet is in direct fluidic connection with the reaction chamber. This is because no nucleic acid extraction, washing, or elution is needed prior to amplification. As before, the cartridge is pierced upon insertion, draining an aliquot of sample into the reaction chamber.

Once enough lysate has wicked into the reaction chamber, the rehydration buffer blister pouch is burst and drained, filling the chamber and mixing reagents (FIG. 12C). The reaction is then heated to an adequate temperature for efficient amplification to occur. Following amplification, the reaction chamber is put into fluidic connection with the lateral flow assay via another wicking channel (FIG. 12D). Once amplicons have reached the sample pad, the running buffer blister is burst, driving amplicons along the lateral flow assay, through the detection strip. The strip results are then read by the user or an automated strip reader. Medical action may be taken based on the strip results.

The diagnostic device may be configured to facilitate communication of health information. Such information may include, for example, location information that allows for tracking, reporting and surveillance of the pandemic. Additionally, the test data may be incorporated into the electronic medical records of the patient for improved patient treatment and follow-up. In some embodiments, barcodes or other indicia may be imprinted on the diagnostic device, allowing the user to photograph the device and results with the barcode in clear view. This photograph can be uploaded to electronic medical record programs, providing diagnostic information to medical workers and/or providing location information for tracking and surveillance.

In another embodiment, the diagnostic device includes a Bluetooth-enabled strip reader or other communication-enabled strip reader. For example, the diagnostic device may allow for the test data to be geotagged and uploaded automatically when connected to a phone or computer via Bluetooth or another communication protocol.

Any suitable DNA polymerase may be included in the nucleic acid amplification reagent. As used herein, the term “DNA polymerase” refers to an enzyme capable of catalyzing the formation of DNA. In embodiments in which the amplification step is performed under isothermal conditions, the DNA polymerase is advantageously a strand-displacing polymerase (i.e., a polymerase with the ability to displace downstream DNA encountered during synthesis). Exemplary strand-displacing DNA polymerases include phi29, Bst, Bsm, Bsu, and Klenow fragment.

As used herein, a “primer” is a nucleic acid designed to bind via complementarity to sequences that flank the target sequence in the template nucleic acid. During amplification, polymerases extend primers. The primer’s binding site should be unique to the target sequence with minimal homology to other sequences to ensure specific amplification of the intended target sequence. In some embodiments, the nucleic acid amplification reagent may comprise at least one primer that is detectably labeled. Exemplary labeling moieties include, without limitation, gold nanoparticles, protein binding ligands, haptens, antigens, fluorescent compounds, dyes, radioactive isotopes, and enzymes. In some embodiments, the nucleic acids are amplified using a labeled primer set (comprising a forward and a reverse primer), generating amplification products with tagged primers at both ends for easy detection via a lateral flow device. Primers may further comprise nuclease cleavage sites and/or blockers (e.g., phosphoramidite blocker) to provide amplification specificity.

In embodiments in which the target nucleic acid is RNA, the eluate may be subjected to a reverse transcription reaction to generate cDNA prior to amplification (i.e., reverse transcription polymerase chain reaction). In these embodiments, the nucleic acid amplification reagent may comprise a reverse transcriptase and at least one suitable primer. A “reverse transcriptase” is an RNA-dependent DNA polymerase. The reverse transcriptase initiates synthesis of a DNA transcript using the RNA as a template, forming a single-stranded cDNA. A double-stranded DNA molecule may be produced from the cDNA using a DNA polymerase. Either the single-stranded cDNA or the double-stranded DNA prepared from reverse transcription may serve as the input for the subsequent amplification reaction.

Standard methods of nucleic acid amplification require (1) high temperatures to increase reaction kinetics and expedite primer-target annealing and (2) expensive laboratory equipment, such as heating blocks, centrifuges, bead-beaters, magnetic beads, and/or volume dispensing robots. To be more suitable for use in a point-of-care device, an amplification method must be free of such requirements. To this end, the devices of the present invention may be configured such that the nucleic acid amplification step is performed isothermally, without the use of a thermocycler. As used herein, “isothermally” or “under isothermal conditions” means that reaction is conducted at a relatively constant temperature. Suitably, the reaction is conducted with temperature fluctuations less than ±10° C., ±5° C., or ±2° C. In some embodiments, the amplification methods are performed without any equipment requiring a power supply to provide source heat for the amplification reaction. The methods are performed at a temperature below 70° C., 65° C., 60° C., 55° C., 50° C., 65° C., or 40° C. In certain embodiments, the methods are performed at a temperature below 37° C. Suitably, the methods may be performed at a temperature between 20° C. and 70° C., 20° C. and 65° C., 20° C. and 60° C., 20° C. and 55° C., 20° C. and 50° C., 20° C. and 45° C., 20° C. and 40° C., or 20° C. and 37° C.

With any isothermal amplification technique, the annealase ICP8 and, optionally, a helicase or a nickase/polymerase may be included to accelerate the reaction. ICP8 is derived from the herpesvirus DNA replication system. This annealase promotes efficient replication of the viral genome during host cell infection by stabilizing single-stranded DNA (ssDNA) and recruiting various factors necessary for replication. Specifically, ICP8 binds ssDNA, samples ssDNA for base pairing, and anneals two ssDNA molecules. Thus, ICP8 can be used to promote the annealing of DNA primers to their complementary targets during an amplification reaction. By increasing reaction kinetics and reducing off-target amplification, ICP8 allows the reaction to be performed at a lower temperature with increased specificity. The ICP8 used with the present invention may be from any available source, including from any herpesvirus or another closely related virus. For instance, the ICP8 may be derived from chelonid herpesvirus 5, a type of herpesvirus that infects the Hawaiian green sea turtle, which has an internal body temperature of 20-25° C.

Since ICP8 lacks helicase function, helicases or nucleases/polymerase must be added to the reaction to generate ssDNA for ICP8 to sample. Any suitable helicase or nuclease/polymerase may be used with the methods of the present invention. Exemplary helicases include, without limitation, UvrD, RecBCD, BLM, WRN, and RecQ. Exemplary nucleases include UL12, nickases, and restriction enzymes.

The largest limitation to the selection of enzymes for use in the amplification reaction will be their ability to function at the temperature at which the devices are intended to be used. However, it is standard practice for one of skill in the art to optimize reaction conditions and enzyme components to achieve particular reaction goals (i.e., sensitivity, specificity, speed, and efficiency at a given temperature). For instance, conditions such as primer length, melting temperature (Tm), and GC content, reaction buffering conditions (e.g., pH, salt concentrations, dNTP concentrations), and crowding agents (e.g., PEG) can be varied. Literature regarding isothermal amplification reactions and ICP8 DNA binding, strand-invasion, and recombination assays may provide guidelines that aid in reaction optimization.

The nucleic acid amplification reagent may further comprise additional components, including cofactors, buffering agents, amplification enhancers, or any combination thereof. As used herein, a “cofactor” is a substance other than the substrate that is essential for the activity of an enzyme. Suitably, the cofactor may be magnesium, which functions as a cofactor for a variety of polymerases. The cofactor may be introduced to the amplification reaction as a salt, e.g., MgSO₄ or MgCl₂. As used herein, a “buffering agent” comprises a weak acid or base used to maintain the acidity (pH) of a solution near a chosen value after the addition of another acid or base. Suitably, the buffering agent may be selected from Tris-HCl, (NH4)₂S0₄, or KC1. As used herein, an “amplification enhancer” is a substance that may enhance amplification specificity, efficiency, consistency, and/or yield. Exemplary amplification enhancers include dimethyl sulfoxide, glycerol, formamide, polyethylene glycol, N,N,N- trimethylglycine (betaine), bovine serum albumin, tetramethylammonium chloride, a detergent, or combinations thereof. Suitably, the detergent is a nonionic detergent such as Tween 20 or Triton X-100.

Any detection device that provides a readout that indicates whether a target nucleic acid is present in a sample may be used with the present invention. The presence of the target nucleic acid may be detected by any suitable method or assay technique, including, without limitation, a binding assay, a colorimetric assay, an electrophoretic assay, a fluorescence assay, a turbidity assay, an electrochemical assay, and the like. Detection devices may provide an analog or digital readout.

In some embodiments, the detection device is a lateral flow device. As used herein, a “lateral flow device” is a porous device capable of detecting the presence of a target nucleic acid sequence traversing a series of beds. Lateral flow devices typically comprise (a) a sample loading area at one end; (b) an area comprising a detectably labelled probe, wherein said detectably labelled probe is not bound to the lateral flow device and is capable of wicking across the lateral flow device; (c) an area comprising a capture probe, wherein said capture probe; and (d) absorbent material, wherein the absorbent material wicks an aqueous sample across the lateral flow device when the aqueous sample is added to the sample loading area. Thus, in some embodiments, the lateral flow device comprises a sample loading area, an amplification area, a solid support, an absorbent sample pad, or any combination thereof. A detailed description of exemplary lateral flow devices can be found in U.S. Pat. Publication No. 2018/0148774, incorporated by reference herein.

In embodiments that utilize a lateral flow device, the test results may be displayed using lateral flow assay (LFA) strips, which provide a readout similar to that of a pregnancy test strip. The strips comprise a capture probe for the target nucleic acid sequence, wherein said capture probe is immobilized on the lateral flow device in a region referred to as the “test area.” The test area can be in any form with well-defined boundaries, such as a dot, or a strip. The capture probe may be immobilized on the lateral flow device by covalent coupling or affinity binding. Suitably, the capture probe is attached to the lateral flow device by biotin:streptavidin affinity binding. Generally, the capture probe is capable of specifically hybridizing to part of the target DNA sequence, separate from the detector probe sequence to which the detectably labelled primer will bind. The LFA strip may comprise multiple probe-capture lines designed to capture different target sequences.

In some embodiments, the amplified RNA or DNA are dual-labeled using two primers that have a biotin label on one primer and a second label (e.g., a FITC, DIGO or TAMRA tag) on the other reverse primer. The streptavidin conjugated AuNPs/latex bead (the colorimetric moiety) will bind to the biotin side of the amplicons while the tag molecule on the other side is captured by an antibody (anti-FITC or anti-DIGO or anti-TAMRA, respectively) attached to the strips. The rest of the streptavidin conjugated AuNPs/latex bead will be captured by the biotin control line on the LFA.

In some embodiments, the detection device is configured such that detection is accomplished by visual inspection, either with or without additional instrumentation. For example, results can be quantified by imaging and analysis with a computer. In some embodiments, the result can be scanned with a smartphone and electronically sent to a clinician, for example, with a computer that has an Adobe Acrobat grayscale converter or an Image J image processing software to quantify the visible light signal from a gold nanoparticle. Likewise, a color wheel for visualization of positive tests may be utilized.

In some embodiments, the devices further comprise a heating element. For use in a point-of-care device, the heating element is advantageously portable and does not require electricity. In particular embodiments, the heating element comprises a battery-powered, cell phone-powered, or solar battery-powered heating film. In alternative embodiments, the heating element may use a reversible or irreversible exothermic chemical reaction to generate heat.

### Kits

The present invention also provides kits comprising any of the compositions or diagnostic devices disclosed herein. The kits may further comprise one or more of a lysis cartridge, a lysis agent, a collection device, a heating element, and a handheld controller. These components may be included to ensure that use of the kit is safe, simple, and hands-off. This is of particular importance when the samples used with the present invention may contain harmful or infectious materials.

In some embodiments, the composition comprising primers for RT-LAMP is provided in a lyophilized form within the kits. For example, the composition may be provided as a dried pellet that is formulated as shown in Table 18.

In some embodiments, the kits include a collection device. The collection device may comprise any suitable device for containing the sample, such as a container, specimen jar, tube, syringe, needle, bag, specimen collection paper, or swab. In some embodiments, the collection device comprises a Puritan® HydraFlock swab or a Pixel nasal self-collection swab, which are designed for absorption and retention of cellular material. Since these swabs can hold approximately 250 µL of sample when fully saturated, their use standardizes sample input without requiring any measurement or transfer of infectious liquids. For saliva samples, the collection device may be a spit tube with or without RNA preservatives. Exemplary spit tubes include, without limitation, Spectrum Solutions LLC SDNA-1000 Saliva Collection Device and OraSure Technologies’ Omnigene Oral OM-505 saliva collection device.

In some embodiments, the kits include a lysis cartridge comprising a lysis agent. Advantageously, the lysis cartridge provides a simple, hands-off means to lyse cellular components within the sample. The lysis cartridge may be configured to allow for direct insertion of a sample (e.g., via a swab or needle). To promote lysis, the operator may be instructed to cap and shake the cartridge. As used herein, a “lysis agent” is a composition capable of breaking down or disrupting a cellular membrane or virus envelope. Ideally, the lysis agent results in efficient cell lysis without the use of any equipment, such as a heating block or vortex. Suitable lysis agents include, without limitation, chaotropic salts (e.g., guanidine thiocyanate, alkali metal perchlorates, alkali metal iodides, NaI, alkali metal trifluoroacetates, alkali metal trichloroacetates, alkali metal thiocyanates, urea, guanidine HCl, guanidine thiocyanate, guanidium thiosulfate, and thiourea), lytic enzymes (e.g., beta glucurondiase, glucanase, glusulase, lysozyme, lyticase, mannanase, mutanolysin, zymolase, cellulase, lysostaphin, pectolyase, and streptolysin O), and detergents (e.g., sodium dodecyl sulfate (SDS), IGPAL-CA630, 3-[(3- cholamidopropyl)dimethylammonio]-1 -propanesulfonate, octyl-β- thioglucopyranoside, octyl-glucopyranoside, 3-(4-heptyl) phenyl 3-hydroxy propyl) dimethylammonio propane sulfonate, 3-[N,N-dimethyl(3-myristoylaminopropyl)ammonio] propanesulfonate, 3- (decyldimethylammonio)propanesulfonate inner salt, 3- (dodecyldimethylammonio)propanesulfonate inner salt, 3-(N,N-dimethylmyristylammonio)propanesulfonate, and n-dodecyl a-D-maltoside). In some embodiments, the lysis agent is a chaotropic salt.

In some embodiments, the lysis cartridge can be used with samples that require particulate filtration, such as stool or sewage (FIG. 7). The lysis cartridge may make use of a small pore filter, such as a filter membrane or a porous foam filter. When lysate is drained from the lysis cartridge, it is forced through the particulate filter through a plunging action. This plunging action resembles how a syringe is evacuated by creating positive pressure within the cartridge chamber, forcing lysate through the filter and into the inlet of the processing/detection device. The filter can also be embedded with resin, activated charcoal or something similar for the removal of non-particulate, chemical contaminants. Complex samples, such as stool, contain reaction inhibitors like bile salts. The removal of such inhibitors can greatly improve nucleic acid recovery and amplification reaction efficiency.

The kits may also include primers and reagents for detecting a control nucleotide sequence. The control nucleotide sequence or primers used to detect it may be any of those disclosed herein.

### EXAMPLES

In the following Examples, the inventors describe the development of a rapid, point-of-care, nucleic acid amplification test (NAAT) for the SARS-CoV-2 virus. The test can detect the virus in upper and lower respiratory specimens. In this test, RNA is extracted using silica microspheres, amplified using reverse transcriptase loop-mediated isothermal amplification (RT-LAMP), and detected by lateral flow assay (LFA) (FIG. 1).

### Example 1. RT-LAMP Amplification and LFA Detection of Synthetic SARS-CoV-2 RNA Targets

The 10X LAMP primer mix for SARS-CoV-2 contains 16 µM each of FIP and BIP, 4 µM of F3 and B3, and 2 µM of LF and LB. The sequence of the primers is listed in Table 10.

TABLE 10

RT-LAMP primer set 1 for SARS-CoV-2 detection

| | | |
|---|---|---|
| Name | Modification | Sequence (5′ to 3′) |
| FIP | | AGGGACATAAGTCACATGCAAGAATTTTTCTTATGTCCTTCCCTCAGT (SEQ ID NO: 52) |
| FIP | FITC tag | FITC-AGGGACATAAGTCACATGCAAGAATTTTTCTTATGTCCTTCCCTCAGT (SEQ ID NO: 52) |
| BIP | | AGAAAAGAACTTCACAACTGCTCCTTTTCAAAGACACCTTCACGAGG (SEQ ID NO: 53) |
| F3 | | GATTTTTGTGGAAAGGGCTATC (SEQ ID NO: 54) |
| B3 | | CAAACCAGTGTGTGCCAT (SEQ ID NO: 55) |
| LF | | GACTACACCATGAGGTGCTG (SEQ ID NO: 56) |
| LB | | CATTTGTCATGATGGAAAAG (SEQ ID NO: 57) |
| LB | Biotin tag | Biotin- CATTTGTCATGATGGAAAAG (SEQ ID NO: 57) |

FITC-tagged FIP and biotin-tagged LB primers are only used to replace non-tagged FIP and LB primers for lateral flow detection. Each 25 µl RT-LAMP reaction mix contains 2.5 µl 10X primer mix, 2.5 µl 10X RT-LAMP reaction buffer (Empirical Bioscience), 3.5 µl 10 mM each dNTPs mix (Empirical Bioscience), 6 µl 100 mM MgSO₄ (Empirical Bioscience), 0.5 µl 15,000 units/ml WarmStart® RTx Reverse Transcriptase (New England Biolabs), 1 µl 8,000 units/ml Bst DNA polymerase (Empirical Bioscience), 0.5 µl 25 µM ROX reference dye (Empirical Bioscience), 1.25 µl 20X EvaGreen dye (Empirical bioscience), and 2 µl of target. The volume is toped up to 25 µl using molecular-biology-grade water. For lateral flow assay detection, the ROX reference dye and EvaGreen dye are not added.

Synthesized RNA target diluted in water or human saliva are used for testing. Human saliva is diluted 10 times using a sample dilution buffer, containing 0.1 mM sodium citrate, 2 mM EDTA to chelate divalent metal cations and inactivate RNAses, and 2.5 mM TCEP. Tris(2-carboxyethyl)phosphine (TCEP) is used to denature RNA-degrading enzymes or RNAses by reducing disulfide bonds in proteins including RNAses. This increases the stability of the RNA and also increases the sensitivity of the assay. This treatment in addition to the heat lysis also improves the safety of the assay for the user by the inactivation of the virions. The diluted saliva is further incubated at 95° C. for 10 min using a heat block before adding to the RT-LAMP reaction mix. For fluorescence detection, the RT-LAMP reaction is carried out at 65° C. for up to 1.5 hours using the QuantStudio® 3 Real-Time PCR System (ThermoFisher Scientific). For lateral flow assay detection, the RT-LAMP mix in 200 µl individual PCR tubes are incubated at 65° C. degrees for 25 min using a water bath. After the incubation, the result is read using the lateral flow assay in EasyNAT® disposable nucleic acid lateral flow assay detection device (Ustar Biotechnologies) following the manufacture’s instruction.

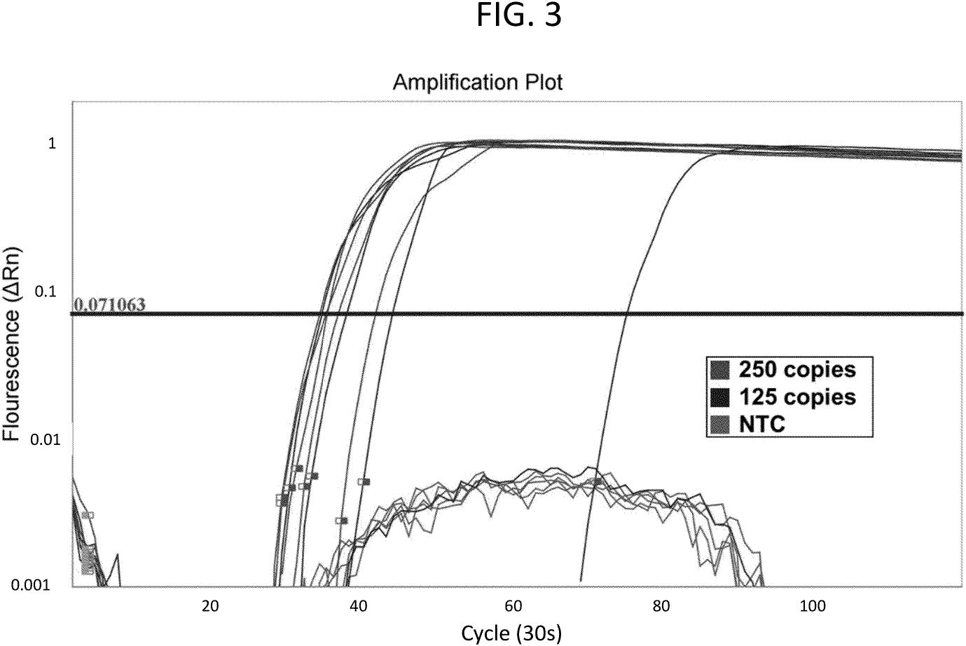

Performance of RT-LAMP detecting synthetic RNA targets. Synthetic RNA targets of 250 copies and 125 copies per reaction were spiked into the RT-LAMP reaction mix. As is shown in FIG. 3, 5/5 of the 250 copies/reaction group and ⅗ of the 125 copies/reaction group were detected within 25 min of incubation. 0/5 of the non-template control was detected in the one-hour incubation period.

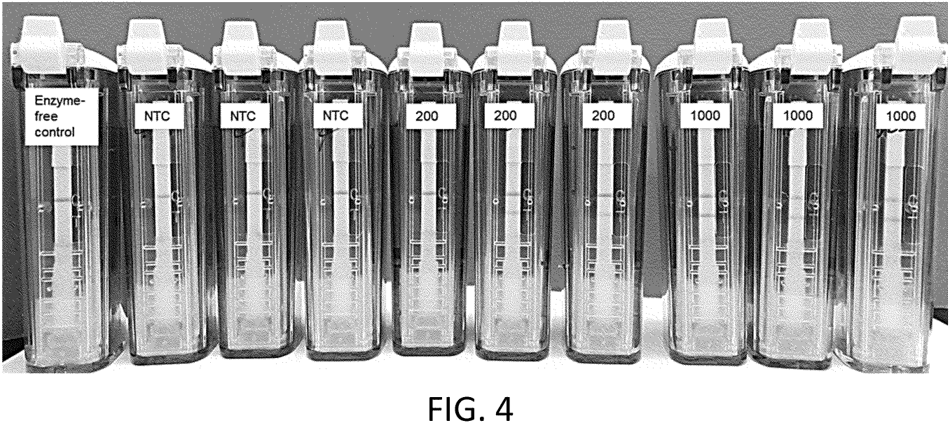

Lateral flow assay detection. RT-LAMP reaction mix was prepared and incubated as described above. An enzyme-free control without sample and enzyme was prepared to check for non-specific binding of the tagged primers. As is shown in FIG. 4, 0/1 of the enzyme-free control and 0/3 of the non-template control was detected, and ⅔ of the 200 copies/reaction and 3/3 of the 1000 copies/reaction were detected after the 25 min incubation period.

### Example 2. RT-LAMP Amplification and LFA Detection of SARS-CoV-2 in Nasopharyngeal Swab Samples

The 10X LAMP primer mix for SARS-CoV-2 contains 16 µM each of FIP and BIP, 4 µM of F3 and B3, and 2 µM of LF and LB. The sequences of the primers are listed in Table 11. The 10X LAMP primer mix for the human control (i.e., a positive control based on detection of the human amylase gene) contains 16 µM each of FIP and BIP, 4 µM of F3 and B3, and 2 µM of LF. The sequences of the primers for detection of this control are listed in Table 12.

TABLE 11

RT-LAMP primer set 2 for SARS-CoV-2 detection

| | | |
|---|---|---|
| Name | Modification | Sequence (5′ to 3′) |
| FIP | | TAGGCAATGATGGATTGACTAGCTATTTTTTATCAGACTCAGACTAATTCTCC (SEQ ID NO: 58) |
| FIP | FITC tag | FITC-TAGGCAATGATGGATTGACTAGCTATTTTTTATCAGACTCAGACTAATTCTCC (SEQ ID NO: 58) |
| BIP | | AACTCTATTGCCATACCCACAAATTTTTTTGGTCATAGACACTGGTAG (SEQ ID NO: 59) |
| F3 | | TTGGTGCAGGTATATGCG (SEQ ID NO: 60) |
| B3 | | ACATTGTACAATCTACTGATGTC (SEQ ID NO: 61) |
| LF | | CACTACGTGCCCGCCGA (SEQ ID NO: 62) |
| LB | | TTTACTATTAGTGTTACC (SEQ ID NO: 63) |
| LB | Biotin tag | Biotin- TTTACTATTAGTGTTACC (SEQ ID NO: 63) |

TABLE 12

RT-LAMP primers for human amylase (control) detection

| | | |
|---|---|---|
| Name | Modification | Sequence (5′ to 3′) |
| FIP | | CCTATGGCTTGGACTTTCCAACTTTTGCTCCTGAACCAGTT GTT (SEQ ID NO: 64) |
| FIP | FAM tag | FAM-CCTATGGCTTGGACTTTCCAACTTTTGCTCCTGAACCAGTT GTT (SEQ ID NO: 64) |
| BIP | Biotin tag | BIO-AGCTTACGTTATCTACCAGAGCATTTTGGTATAAATGCGA ACCGCT (SEQ ID NO: 65) |
| F3 | | ATCTAGAGGCTGGGAAGG (SEQ ID NO: 66) |
| B3 | | AGTGTCCTTTCCAGAAACT (SEQ ID NO: 67) |
| LF | | CCTGACAGACCGACAAGACGGA (SEQ ID NO: 68) |

Each 30 µl COVID RT-LAMP master mix contains 5 µl 10X primer mix, 5 µl 10X RT-LAMP reaction buffer (Empirical Bioscience), 7 µl 10 mM each dNTPs mix (Empirical Bioscience), 3.5 µl 100 mM MgSO₄ (Empirical Bioscience), 1 µl 15,000 units/ml WarmStart® RTx Reverse Transcriptase (New England Biolabs), and 1.75 µl 8,000 units/ml Bst DNA polymerase (Empirical Bioscience). The volume is toped up to 30 µl using molecular-biology-grade water.

Each 30 µl human control LAMP master mix contains 5 µl 10X primer mix, 5 µl 10X RT-LAMP reaction buffer (Empirical Bioscience), 7 µl 10 mM each dNTPs mix (Empirical Bioscience), 3.5 µl 100 mM MgSO₄ (Empirical Bioscience), and 2 µl 8,000 units/ml Bst DNA polymerase (Empirical Bioscience). The volume is toped up to 30 µl using molecular-biology-grade water.

The inactivation buffer contains 1 M TCEP, 0.25 M guanidinium hydrochloride, and 0.125 M EDTA at pH 7.6. The dilution buffer contains 1 mM sodium citrate at pH 6.5. The negative control mix contains 17.85 mM TCEP, 4.46 mM guanidium hydrochloride, 2.23 mM EDTA, and 0.89 mM sodium citrate at pH 6.5. The positive control mix contains 17.85 mM TCEP, 4.46 mM guanidium hydrochloride, 2.23 mM EDTA, 0.89 mM sodium citrate, and 200 copies/µl synthetic SARS-CoV-2 RNA (Twist Bioscience) at pH 6.5.

Detection of synthetic SARS-CoV-2 RNA from contrived samples. Contrived samples were made by adding 2 µl of 1.125 X 10⁴ copies/µl of synthetic SARS-CoV-2 RNA into 20 µl of inactivation buffer immediately followed by adding 100 µl of COVID-19-negative nasopharyngeal swab sample in UTM (Universal Transport Medium) to prevent RNA degradation by RNase. The RNA, inactivation buffer, and sample were mixed by pipetting five times. The inactivated sample was incubated at 95° C. degrees for 5 min using a heat block to mimic the virus lysis step. After incubation, 1 ml of dilution buffer was added to the sample and mixed by pipetting five times. A 20 µl aliquot of diluted sample was then added to the 30 µl COVID RT-LAMP master mix and mixed by pipetting five times. Another 20 µl aliquot of sample was added to the 30 µl human control LAMP master mix and mixed by pipetting for five times. Before and after each batch of sample testing, a positive control and a negative control COVID RT-LAMP reaction mix were made by mixing a 20 µl of positive control mix or negative control mix respectively with a 30 µl COVID RT-LAMP master mix by pipetting five times.

The prepared COVID RT-LAMP reaction mix and human control LAMP reaction mix were incubated at 65° C. for 25 min using a water bath. After the incubation, the test result was read using the lateral flow assay in EasyNAT® disposable nucleic acid lateral flow assay detection device (Ustar Biotechnologies) following the manufacture’s instruction. The COVID-19 test was able to detect 19/20 of the contrived samples at 225 copies/µl synthetic SARS-CoV-2 RNA, and 20/20 human control test were positive for the nasopharyngeal swab sample in UTM.

### Example 3. Standard Operating Procedure for RT-LAMP Amplification and LFA Detection of SARS-CoV-2 in Nasopharyngeal Swab Samples

Materials:
Heat block set to 95° C. degrees
Water bath set to 65° C. degrees
BioUSTAR EasyNAT® test
P1000 and P100 pipettors and tips

Preparation:
1. Prepare the inactivation buffer: TCEP (tris(2-carboxyethyl)phosphine) 1 M, guanidine hydrochloride 0.25 M, EDTA 0.125 M, adjust pH using 5 N NaOH to 7.6, aliquot into 20 µl single use aliquots in 1.7 ml microcentrifuge tubes, store at -20° C.
2. Prepare the dilution buffer: Sodium citrate buffer 1 mM, pH = 6.5, aliquot into 1 ml single use aliquots in 1.7 ml microcentrifuge tubes, store at -20° C.
3. Prepare the negative control mix (20 µl per control): mix 20 µl inactivation buffer with 100 µl molecular biology grade water and 1 ml dilution buffer, aliquot into 50 µl aliquots in 0.65 ml microcentrifuge tubes, store at -80° C.
4. Prepare the positive control mix (20 µl per control): mix 1 µl of 10⁵ copies/µl COVID 19 control RNA with 500 µl negative control mix, aliquot into 50 µl aliquots in 0.65 ml microcentrifuge tubes, store at -80° C.
5. Prepare SARS-CoV-2 RT-LAMP 10X primer mix as detailed in Table 13.

TABLE 13

RT-LAMP 10X SARS-CoV-2 primer mix

| | | |
|---|---|---|
| FIP | 16µM | FAM-TAGGCAATGATGGATTGACTAGCTATTTTTTATCAGACTCAGACTAATTCTCC (SEQ ID NO: 58) |
| BIP | 16µM | AACTCTATTGCCATACCCACAAATTTTTTTGGTCATAGACACTGGTAG (SEQ ID NO: 59) |
| F3 | 2µM | TTGGTGCAGGTATATGCG (SEQ ID NO: 60) |
| B3 | 2µM | ACATTGTACAATCTACTGATGTC (SEQ ID NO: 61) |
| LF | 4µM | CACTACGTGCCCGCCGA (SEQ ID NO: 62) |
| LB | 4µM | BIO-TTTACTATTAGTGTTACC (SEQ ID NO: 63) |

6. Prepare human control RT-LAMP 10X primer mix as detailed in Table 14.

TABLE 14

RT-LAMP 10X control primer mix

| | | |
|---|---|---|
| FIP | 16µM | FAM-CCTATGGCTTGGACTTTCCAACTTTTGCTCCTGAACCAGTTGTT (SEQ ID NO: 64) |
| BIP | 16µM | BIO-AGCTTACGTTATCTACCAGAGCATTTTGGTATAAATGCGAACCGCT (SEQ ID NO: 65) |
| F3 | 2µM | ATCTAGAGGCTGGGAAGG (SEQ ID NO: 66) |
| B3 | 2µM | AGTGTCCTTTCCAGAAACT (SEQ ID NO: 67) |
| LF | 4µM | CCTGACAGACCGACAAGACGGA (SEQ ID NO: 68) |

7. Prepare COVID test RTLAMP reaction mix (30 µl per reaction) as detailed in Table 15. Aliquot in 200 µl individual PCR tubes and store at -20° C.

TABLE 15

RT-LAMP reaction mix

| | |
|---|---|
| Primer mix | 5 µl |
| RTLAMP reaction buffer (Empirical Bioscience) | 5 µl |
| 10 mM each dNTPs mix (Empirical Bioscience) | 7 µl |
| 100 mM MgSO₄ (Empirical Bioscience) | 3.5 µl |
| 8 U/µl Bst polymerase (Empirical Bioscience) | 1.75 µl |
| 15 U/µl WarmStart® RTx Reverse Transcriptase (New England Biolabs) | 1 µl |
| Molecular biology grade water | to 30 µl |

8. Prepare Human control LAMP 10X reaction mix as detailed in Table 16. Aliquot in 200 µl individual PCR tubes and store at -20° C.

TABLE 16

RT-LAMP human control 10x reaction mix

| | |
|---|---|
| Primer mix | 5 µl |
| RTLAMP reaction buffer (Empirical Bioscience) | 5 µl |
| 10 mM each dNTPs mix (Empirical Bioscience) | 7 µl |
| 100 mM MgSO₄ (Empirical Bioscience) | 3.5 µl |
| 8 U/µl Bst polymerase (Empirical Bioscience) | 2 µl |
| Molecular biology grade water | to 30 ul |

Protocol:
1. Thaw out reagents and warm up heat block and water bath. NP swab in transport media, such as the UTM Viral Transport (COPAN Diagnostics Inc, CAT# 3C047N), BD UVT Viral Transport Collection Kit (BD CAT# 220531) or an equivalent, should be used as sample input. A positive control and a negative control should be run at the beginning and the end of each batch of testing.
2. Take 100 µl of sample and dispense into the microcentrifuge tube containing 20 µl inactivation buffer using a fixed volume dropper, pipette in and out five times to mix.
3. Place the tube on the heat block for heat lysis at 95° C. for 5 min. If the tube used will pop open during/after heat lysis, cap locks should be used to prevent that.
4. Remove the tube from the heat block, dispense a 1 mL pre-filled volume of dilution buffer into the inactivated sample, pipette in and out five times to mix.
5. Take 40 µl of the diluted sample using a fixed volume dropper, dispense into the PCR tube containing a lyophilized COVID RT-LAMP reaction mix, pipette in and out five times to mix.
6. Take another 40 µl from the diluted sample, dispense into the PCR tube containing a lyophilized human control RT-LAMP reaction mix, pipette in and out five times to mix.
7. For controls, take 20 µl from the positive or negative control mix, dispense into the PCR tube containing COVID RT-LAMP reaction mix, pipette in and out five times to mix.
8. Place the PCR tubes in the water bath to incubate at 65° C. for 25 min, a floater with holes of appropriate size should be used.
9. After the incubation, take the PCR tubes out to read the result using the BIOUSTAR EasyNAT® following the manufacturer’s instructions. The test result should be ready within 1 min of the insertion into the device.
10. Control samples that can be used with this protocol:
   a) A no template (negative) control is needed to ensure that there is no amplicon contamination in the environment that can result in false positives, and the master mix components and amplification are not causing primer dimerization that can lead to sample-independent false amplification and is used before and after of each batch of testing series and/or shift together with actual COVID-19 sample tests. It is a mixture of inactivation buffer, dilution buffer, and molecular-biology-grade water.
   b) A positive template control of synthetic SARS-CoV-2 RNA (MT007544.1, Twist Bioscience) with a concentration of 200 copies/µL (equivalent to 10X LOD when added to the control reaction tubes) is needed to ensure that the reverse transcription and amplification are taking place, and that the primers are working and amplifying at the lowest level of detection. The positive control covers the whole genome of SARS-CoV-2 and contains all the targets of the probes used in the assay. Positive control test is used before and at after of every testing series and or shift.
   c) An extraction control is not needed since the test does not require RNA extraction.
   d) A specimen control of human amylase gene is needed to ensure that the polymerase is functional and that the sample does not contain enough of any LAMP inhibitor to inhibit the reaction, and that sufficient quantities of the human sample was obtained to amplify gene in the human NP swab sample and is used with every sample tested. The internal control is amplified and detected as a separate reaction in a separate lateral flow assay contained in a cartridge and is run on every sample in parallel to the RT-LAMP test for the virus.

Results:
The T line detects SARS-CoV-2 RNA in the case of the COVID-19 test and detects human DNA in the case of the human specimen control test (see FIG. 14).
The C line only detects the lateral flow control; it does not detect the quality controls. (see FIG. 14).

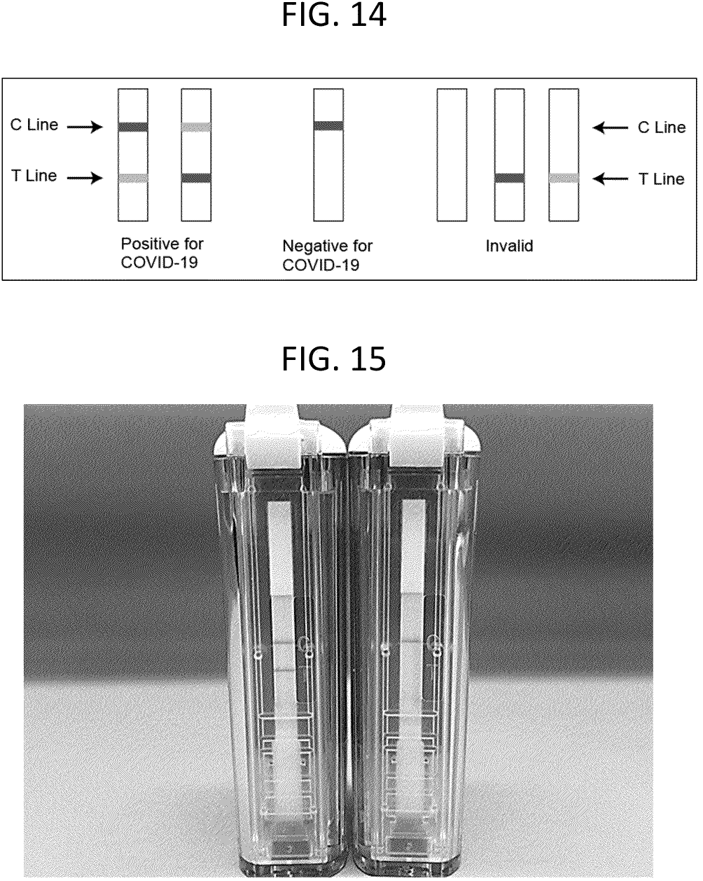

Controls:
Negative Control - The no template negative control should have a visible C line, and no visible T line (see FIG. 15).
Positive Control - The positive template control should have a visible C line and a visible T line (see FIG. 15).
Internal Control - Each clinical sample must have a visible C line and a visible T line for the human gene control to confirm that the test is valid (see FIG. 15).

Interpretation:
One or both positive control(s) is(are) negative: all tests are invalid
One or both negative control(s) is(are) positive: all tests are invalid
Both of positive controls are positive, and both of negative controls are negative:
   i. COVID test is positive and human control is positive: positive result
   ii. COVID test is negative and human control is positive: negative result
   iii. COVID test is positive and human control is negative: test is invalid
   iv. COVID test is negative and human control is negative: test is invalid

Components: In some embodiments, the COVID test reagents are packaged as a kit. Examples of components that could be included in such kits are listed in Table 17.

TABLE 17

Exemplary components for COVID test kit

| | |
|---|---|
| Component | Volume |
| Hand-held lateral flow assay cartridge | N/A |
| Instructions for Use | N/A |
| Lysis or Inactivation Buffer | 0-100 mL |
| LAMP Bst II Pol Enzyme | 0-2 mL |
| 10x RTLAMP Buffer | 0-2 mL |
| Reverse Transcriptase Enzyme | 0-2 mL |
| Primers for SARS-CoV-2 | 0-2 mL |
| Human control primers | 0-2 mL |
| Deoxyribonucleotide phosphate (dATP, dCTP, dGTP, dTTP) | 0-2 mL |
| Enzyme co-factor salt, e.g., magnesium sulfate | 0-2 mL |
| Dilution buffer | 0-2 mL |
| Positive control RNA | 0-2 mL |
| Molecular grade H₂O (Negative Control) | 0-2 mL |
| 100 µL transfer pipettes | N/A |
| 40 µL transfer pipettes | N/A |
| General purpose transfer pipettes | N/A |

In some embodiments, the SARS-CoV-2 RT-LAMP reaction mix and the control RT-LAMP reaction mix are provided as dried (e.g., lyophilized) pellets. The pellets may be formulated as shown in Table 18.

TABLE 18

Formulations of prepared reaction mixes

| | |
|---|---|
| Reagent | Components |
| COVID reaction mix | 10X RT-LAMP reaction buffer |
| | COVID RT-LAMP primers |
| | Warmstart RTx reverse transcriptase |
| | Bst II polymerase |
| | Deoxyribonucleotide phosphate (dATP, dCTP, dGTP, dTTP) mix |
| | Magnesium sulfate |
| Specimen control reaction mix | 10X RT-LAMP reaction buffer |
| | Human amylase LAMP primers |
| | Bst II polymerase |
| | Deoxyribonucleotide phosphate (dATP, dCTP, dGTP, dTTP) mix |
| | Magnesium sulfate |

Testing capabilities: The test takes approximately 35 minutes from start to test read out, as detailed below. This is a medium throughput test that requires no sophisticated equipment. One operator can perform up to 10 tests per hour in batches. Specimen transport to results will vary from site to site and it is dependent on the technical team conducting the assay. Ideally, the tests will be conducted in series of at least 30 tests at the time to increase throughput and time to result. The test is also ideal for rapid, one-off testing without the need for batching e.g., at a rural hospital or urgent care before a procedure.

Estimated testing time:
1) Add sample to lysis/storage buffer and heat lysing: 5 minutes
2) Perform thermal cycler amplification: 25 minutes
3) Set up cassette and obtain read out: 5 minutes

### Example 4. Performance Evaluation of the COVID Diagnostic Test Described in Example 3

### 1. Limit of Detection (LoD) - Analytical Sensitivity

The limit of detection (LOD) of the COVID-19 test described in Example 3 was estimated based on the lowest concentration of a synthetic SARS-CoV-2 RNA (Twist Synthetic SARS-CoV-2 RNA, MT007544.1) spiked into clinical COVID-19 negative nasopharyngeal swab (NP) matrixes that could be detected in 19 out of 20 replicates. A preliminary determination of the LOD was performed by two three-fold dilutions of the synthetic RNA targets into negative NP swabs with three replicates in each dilution group. The detection result of the preliminary LOD determination is summarized in Table 19 and the full data set can be found in Appendix 2. The lowest diluted concentration (225 copies/µL) that can be detected 3/3 replicates was further confirmed by the successful detection of 19/20 replicates to be the LOD of the COVID-19 test, as indicated in Table 19.

TABLE 19

Summary of LOD determination of the COVID-19 test

| Dilution group | 2025 copies/µL | 675 copies/µL | 225 copies/µL |
|---|---|---|---|
| Detection rate | 3/3 | 3/3 | 3/3 |
| Detection rate | | | 19/20 |

### 2. Inclusivity (analytical Sensitivity)

The inclusivity of the COVID-19 test primer set was evaluated via in silico analysis using NC_045512 from GenBank as a reference for SARS-CoV-2. In total, 22,427 complete length SARS-CoV-2 genomes were downloaded from the NCBI database (Oct. 23, 2020) and aligned. Before analysis, 18 sequences were excluded due to ambiguous nucleotides in target region, which reduced the total to 22,409 sequences for inclusivity check. Results are summarized in Table 20.

TABLE 20

In silico inclusivity analysis of 22,409 SARS-CoV-2 sequences

| Primer name | F3 | B3 | FIP (F1c+F2) | F1c | F2 | BIP (B1c+B2) | B1c | B2 | LF | LB |
|---|---|---|---|---|---|---|---|---|---|---|
| Primer length nucleotides | 18 | 23 | 49 | 25 | 24 | 44 | 24 | 20 | 17 | 18 |
| % 0 mismatches | 99.94% | 99.94% | 98.80% | 99.62% | | 99.18% 98.44% | 98.60% | 99.84% | 99.85% | 99.18% 99.61% |
| 0 mismatches | 22,395 | 22,396 | 22,141 | 22,323 | 22,226 | 22,060 | 22,095 | 22,373 | 22,376 | 22,322 |
| 1 mismatch | 14 | 13 | 267 | 86 | 183 | 348 | 314 | 36 | 33 | 87 |
| 2 mismatches | 0 | 0 | 1 | 0 | 0 | 1 | 0 | 0 | 0 | 0 |
| ≥3 mismatches | 0 | 0 | 0 | 0 | 0 | 0 | 0 | 0 | 0 | 0 |

No isolate had an individual primer region had more than 1 SNV, 18 isolates (0.08%) had 2 primer regions with 1 SNV (of those 18, 1 isolate had 1 SNV in each region of FIP, a 2nd isolate had 1 SNV in each region of BIP). Previous work has demonstrated that a single nucleotide mismatch typically has no impact on the limit of detection of LAMP assays (PMID 25103205).

In summary, in silico analysis predicted that the COVID-19 test could detect all SARS-CoV-2 strains.

### 3. Cross-Reactivity (Analytical Specificity)

All primers were analyzed via BLASTn analysis queries of the nucleotide collection (nt) database on Oct. 23, 2020 filtered with the taxonomy identifiers of the high priority organisms (see Appendix 3 for detailed information). BLASTn was run individually for every organism. Search parameters were automatically adjusted for short input sequences. Additional settings included: expect threshold 1000, match score 1 and mismatch scores -3, and penalty to create gap in an alignment 5 and extend a gap in an alignment 2. For SARS-CoV-1 the BLASTn (NCBI: txid694009) excluded SARS-CoV-2 (taxid: 2697049) and bat coronaviruses, microbial interference is unlikely from Bat SARS because they are not common human pathogens.

There were no priority organisms with more than 1 primer over 80% threshold. Since LAMP requires 6 primers and as such amplicon generation is not possible with a single primer it is not expected that these microorganisms will be amplified or produce cross-reactive signal. Therefore, we did not perform wet testing of cross reactivity to other pathogens.

The full list of organisms with one primer over 80% similarity is:
F3: Pseudomonas aeruginosa, Chlamydia trachomatis
B2: bat adenovirus (Sequence ID: KX961096.1, highest human adenovirus is 60% similarity)
LF: Mycobacterium tuberculosis, Bordetella pertussis, Bacillus anthracis, Staphylococcus epidermidis, Staphylococcus aureus
LB: Influenza A (H6N1 Sequence ID: EU049951.1, next highest influenza A sequence is 72% similarity), Streptococcus pneumoniae, Candida albicans)

### 4. Interference Study

Interfering substances studies were performed using clinical COVID-19 negative NP matrices spiked with and without synthetic SARS-CoV-2 RNA at a concentration of 3X LOD. The interfering substances were added to the positive or negative contrived samples at the indicated concentrations showing in Table 21. Each substance was tested at the highest medically relevant concentration in three replicates for both positive and negative contrived samples. Results indicates that the COVID-19 test can tolerate the presence of potential interfering substances listed in Table 21.

TABLE 21

Summary of interfering substance testing of the COVID-19 test

| Interfering substance | Active ingredients | Spiked concentration | Positive sample (3X LOD) | Negative sample |
|---|---|---|---|---|
| Whole blood | Whole blood | 2.5%(v/v) | 3/3 | 0/3 |
| Mucin | Mucin | 2.5 mg/ml | 3/3 | 0/3 |
| Ethanol (70%) | Ethanol | 2.5%(v/v) | 3/3 | 0/3 |
| Nasal allergy spray (Nasocort) | Triamcinolone acetonide | 10%(v/v) | 3/3 | 0/3 |
| Equate Nasal congestion spray (Afrin) | Oxymetazoline HCl | 10%(v/v) | 3/3 | 0/3 |
| Nyquil cold & flu | Acetaminophen, Dextromethorphan HBr, Doxylamine succinate | 2.5%(v/v) | 3/3 | 0/3 |
| Equate allergy relief nasal spray | Fluticasone propionate | 10%(v/v) | 3/3 | 0/3 |
| Emergen-C | Zinc, Magnesium, Riboflavin, Vitamin C | 5 mg/ml | 3/3 | 0/3 |
| Equate Saline nasal spray | NaCl, Phenylcarbinol, Nemalkonium Chloride | 10%(v/v) | 3/3 | 0/3 |
| Chloraseptic spray | Phenol, Glycerin | 10%(v/v) | 3/3 | 0/3 |
| Tamiflu | oseltamivir | 2.5 µg/ml | 3/3 | 0/3 |
| Zinc tablets | Zincum Gluconicum | 5 mg/ml | 3/3 | 0/3 |
| Mupirocin | mupirocin | 5 mg/ml | 3/3 | 0/3 |
| Cepacol sore throat | Benzocaine, Dextromethorphan HBr | 5 mg/ml | 3/3 | 0/3 |

### 6. Biotin Interference Study

Since the COVID-19 test uses biotin technology in its lateral flow detection step, the highest concentration of biotin present in samples that will not cause a false negative result was investigated. Four three-fold dilutions of biotin spiked into clinical COVID-19 negative NP matrixes with synthetic SARS-CoV-2 RNA at a concentration of 3X LOD with 3 replicates in each dilution groups were tested for false negative. Negative contrived samples without SARS-CoV-2 RNA at the highest biotin concentration were tested with 3 replicates to rule out false positive result. After the preliminary determination, the highest concentration of biotin that did not cause any false negative result was confirmed by the successful detection of 19/20 replicates with synthetic SARS-CoV-2 RNA spiked in at 1XLOD. The results (Table 22) indicate the COVID-19 test can tolerate as high as 15 µg/ml biotin present in samples.

TABLE 22

Summary of biotin interfering substance testing of the COVID-19 test

| Biotin Concentration | | 1200 µg/ml | 400 µg/ml | 133 µg/ml | 44 µg/ml | 15 µg/ml |
|---|---|---|---|---|---|---|
| Contrived sample | 3XLOD | 0/3 | 0/3 | 0/3 | 0/3 | 3/3 |
| detection rate | 1XLOD | | | | | 19/20 |
| | Negative | 0/3 | | | | |

### 6. Clinical Evaluation

A clinical study of the COVID-19 test was performed. This study evaluated a total of 60 (30 positive and 30 negative) individual, de-identified, residual nasopharyngeal swab (NPS) specimens collected under IRB approval during routine examination of patients. A single replicate of each blinded sample was tested in the clinical laboratory, and positive and negative agreement were based on the comparator result. The specimens were previously tested using another Emergency Use Authorization (EUA) authorized molecular test, the Roche cobas SARS-CoV-2 assay (the comparator). The positive percent agreement (PPA) was 90% and the negative percent agreement (NPA) was 100%. No invalid results were obtained during this study. The results are summarized in Table 23.

TABLE 23

Summary of clinical evaluation of the COVID-19 test

| | | Comparator Test | | |
|---|---|---|---|---|
| | | Positive | Negative | Total |
| COVID-19 Test | Positive | 27 | 0 | 27 |
| | Negative | 3 | 30 | 33 |
| | Total | 30 | 30 | 60 |

PPA: 90%; 95% CI (72.3%, 97.4%)
NPA: 100%; 95% CI (85.9%, 100%)

### Example 5. Amplification Kit Components

### Materials

1. 10X Primer Mix
2. NEB WarmStart® LAMP Kit
3. RNase-free water, stored in single use aliquots
4. Saliva collection buffer: 0.1 mM sodium citrate and 2 mM EDTA at pH 9.6, stored in 800 µL single use aliquots in 1.5 mL RNase-free Eppendorf tubes
5. 1.5 mL RNase-free Eppendorf tube cap holders
6. Heating block

### 10X Primer Mix

A 10X primer mix will be made in single use aliquots (100 µL) for each primer set as follows:
1. 16 µL forward internal primer (FIP) at 100 µM,
2. 16 µL backward internal primer (BIP) at 100 µM,
3. 4 µL forward 3 primer (F3) at 100 µM,
4. 4 µL backward 3 primer (B3) at 100 µM,
5. optionally, 2 µL loop forward primer (LF) at 100 µM,
6. optionally, 2 µL loop backward primer (LB) at 100 µM, and
7. balance RNase free water.

### Lysis Buffer

Exemplary lysis buffers include the following:
1. 1 mM sodium citrate, 2.5 mM TCEP, 2 mM EDTA, and 0.1%IGPAL-CA630;
2. 1 mM sodium citrate, 2.5 mM TCEP, and 2 mM EDTA;
3. 1 mM sodium citrate, 2.5 mM TCEP, and 1 mM EDTA; or
4. 1 mM sodium citrate, 2.5 mM TCEP.

Suitably, the buffers may be pH adjusted, for example to 6.5.

### Other Buffers

1. Inactivation buffer: 1 M TCEP, 0.25 M guanidium hydrochloride, and 0.125 M EDTA at pH 7.6, stored in 20 µL single use aliquots in 1.5 mL RNase-free Eppendorf tubes
2. Dilution buffer: 1 mM sodium citrate at pH 6.5

### Example 6. Kit Instructions for RT-LAMP Amplification and LFA Detection of SARS-CoV-2 in Nasopharyngeal Swab Samples

Kit components:
Tube 1: used for sample preparation, contains drop of Inactivation Buffer
Tube 2: contains Dilution Buffer
Reaction Tubes with lyophilized amplification reagents:
   Tube 3: contains COVID-19 reaction chemicals
   Tube 4: contains specimen control reaction chemicals
Liquid droppers: Dropper A (100 µL), Dropper B (general purpose), and Dropper C (40 µL)
BioUSTAR EasyNAT amplicon detection devices in foil pouches
Cardboard tube rack to hold tubes upright during sample processing

Tools needed:
miniPCR® mini16 thermal cycler (SKU: QP-1016-01)
Amazon Fire7 16 GB tablet (amazon.com)
Proper PPE (gloves, gown, mask, and goggles)
Permanent marker for labeling tubes

Note: Sample input should only be nasal or NP swab in UTM or equivalent virus transport media. Protocol:

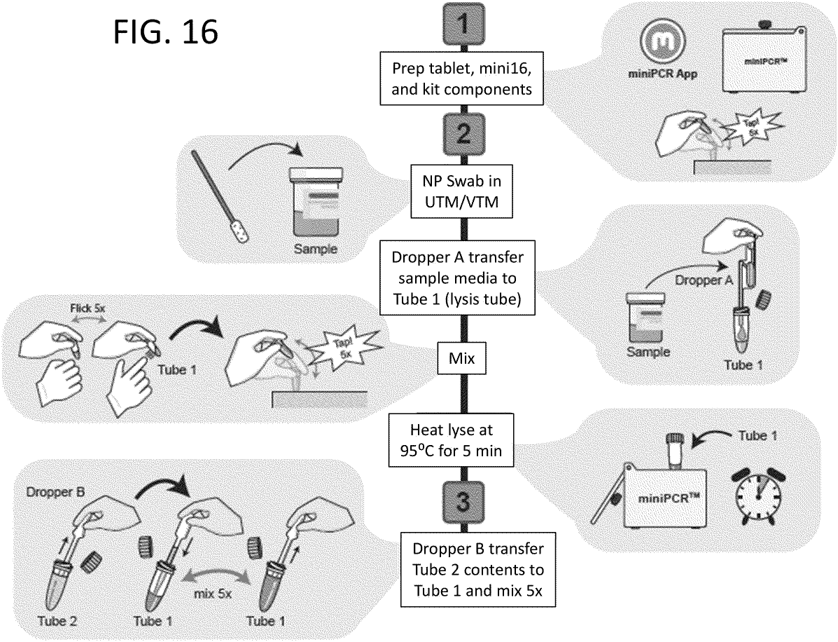

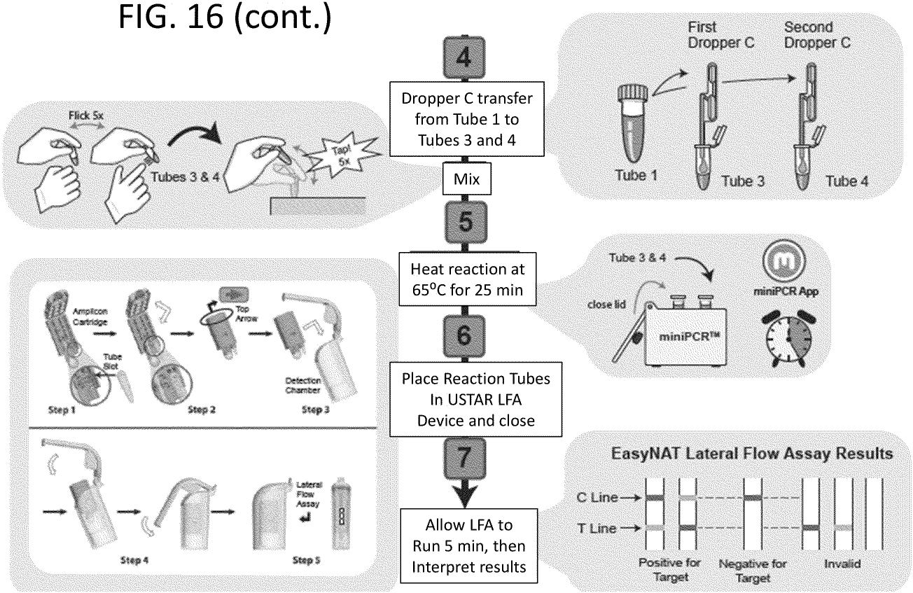

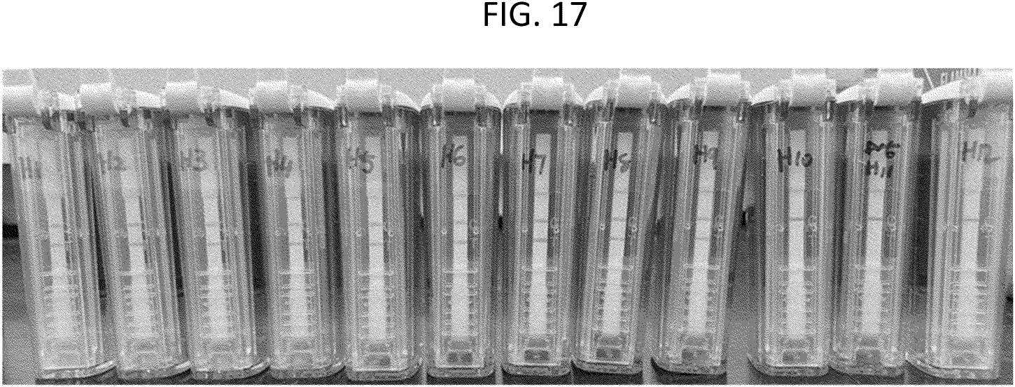

*See FIG. 16 for a schematic depiction of this protocol.

### Step 1. Prepare Kit and Lab Space

The Fire7 tablet purchased from GoDx will have the miniPCR App (Ampylus LLC) preinstalled. Assay heating protocols, GoCOVIDx Program 1 and Program 2, will already be loaded into the miniPCR App. Within the miniPCR App, connect the tablet to the machine via Bluetooth™. Larger 1.5 mL tubes (Tube 1) fit into center channel junctions of miniPCR machine. Smaller 0.2 mL tubes (Tube 3 & 4) fit into the miniPCR tube slots.

Prepare kit components. For each sample, unpack the following, placing tubes in tube rack:
One Tube 1 for sample preparation (contains Inactivation Buffer)
One Tube 2 (contains Dilution Buffer)
Reaction Tubes - one Tube 3 (COVID-19 reaction), one Tube 4 (specimen control reaction)
Liquid transfer pipettes - one Dropper A, one Dropper B, and two Dropper Cs
2 BioUSTAR EasyNAT amplicon detection devices

[Note: during shipping, reagents move around in tubes. Collect all contents into bottom of each tube by tapping the bottom of each tube onto a countertop five times.]

### Step 2. Inactivate and Heat Lyse

Squeeze the top bulb of Dropper A, then immerse dropper tip into sample media. Release pressure on bulb to pick up a portion of sample.
Transfer the liquid in Dropper A to Tube 1 by putting the tip of the dropper into the tube and squeezing the top bulb to release the liquid.

[Note: Droppers A and C are specifically designed to transfer specific volumes via the pipette tube. Extra liquid in the overflow bulb is OK. There is no need to squeeze the overflow bulb.]

Firmly cap Tube 1 and mix its contents using the tube-flick method:
Hold the top of the tube in one hand and gently flick the tube bottom 5 times with the other hand.
Tap Tube 1 five times on the countertop to collect liquid at bottom of the tube.
Place mixed Tube 1 upright in one of the center channel junctions cut into the mini16. Leave lid of mini16 open for this step.
Using the miniPCR App on the Fire7, run GoCOVIDx Program 1, heating Tube 1 contents to 95° C. for 5 minutes.
When the alarm sounds, remove the tube from mini 16 and proceed to step 3. The mini 16 will rapidly cool once the program 1 ends.

### Step 3. Dilute Lysate

Squeeze bulb of Dropper B then immerse tip into Tube 2 liquid. Release pressure on bulb to pick up entire volume and dispense into Tube 1 by moving tip into Tube 1 and squeezing the bulb.
Keeping dropper tip at the bottom of Tube 1, gently mix the liquids by releasing then squeezing the bulb 5 times.
After mixing, fully expel liquid into Tube 1 and discard dropper.

### Step 4. Mix Sample Reactions

Each Tube 3 & 4 contains a dried-down pellet of reaction chemicals for either COVID-19 or specimen control (check tube label). The dried down pellet will dissolve when liquid is added to the tube in the following steps.
Squeeze the top bulb of the first Dropper C then put the dropper tip into the liquid in Tube 1. Release pressure on the top bulb to pick up a portion of the liquid.
Move the tip of Dropper C into Tube 3 and squeeze the liquid onto the dried pellet.
Firmly cap Tube 3 and discard first Dropper C.
Using the second Dropper C, collect another portion of the Tube 1 liquid, but this time squeeze the liquid onto the dried pellet in Tube 4.
Firmly cap Tube 4 and discard second Dropper C.
Mix Tube 3 & 4 reaction solutions using the tube-flick method.
Following mixing, be sure to collect all Tube 3 & 4 contents into tube bottom using tapping method.

### Step 5. Incubate Amplification Reactions

Place all reaction tubes (Tube 3 & 4) prepared in Step 4 into miniPCR tube slots to run amplification reaction.
Using the miniPCR App on the Fire 7, run GoCOVIDx Program 2, heating tube contents to 65° C. for 25 minutes. This time, snap the lid closed.
An alarm sounds once amplification incubation is complete, the mini16 will rapidly cool to stop the reaction.

Important: DO NOT OPEN these tubes! From this point forward the tubes must remain tightly capped shut.

### Step 6. Run LFA Detection Assay

Prepare two BioUSTAR EasyNAT amplicon detection devices.
Open BioUSTAR EasyNAT foil pouch and organize contents.
Contents include an amplicon cartridge and a detection chamber.

Place Tube 3 into one EasyNAT device and Tube 4 into the other.

Follow the instructions below to perform the detection assay:
1. Place tube into the tube slot of the amplicon cartridge.
2. Snap the cartridge shut, securing the tube and buffer bulb.
3. Orient each amplicon cartridge properly by locating the arrow on top of closed cartridge, tube protruding out the bottom. Insert the cartridge, bottom-first, into the detection chamber with the arrow pointing toward the lateral flow strip (longest flat edge).
4. Firmly snap the detection chamber shut, ensuring that the lid is locked.
5. Allow the lateral flow assay to run for five minutes before interpreting results.

### Step 7. Interpret Results and Report

The C line only detects flow control, it does not detect quality controls. If the C Line fails to appear, the lateral flow test results are invalid.
The T line detects target material: Tube 3 target determines the COVID-19 diagnosis, and Tube 4 target confirms that adequate patient sampling was performed.
If Tube 4 target is not detected, test results are invalid due to poor sampling.
Any pink- to red-colored T line should be recorded as a positive (+) result and an absence of a line should be recorded as a negative (-) result.

### Example 7. Exemplary Diagnostic Device

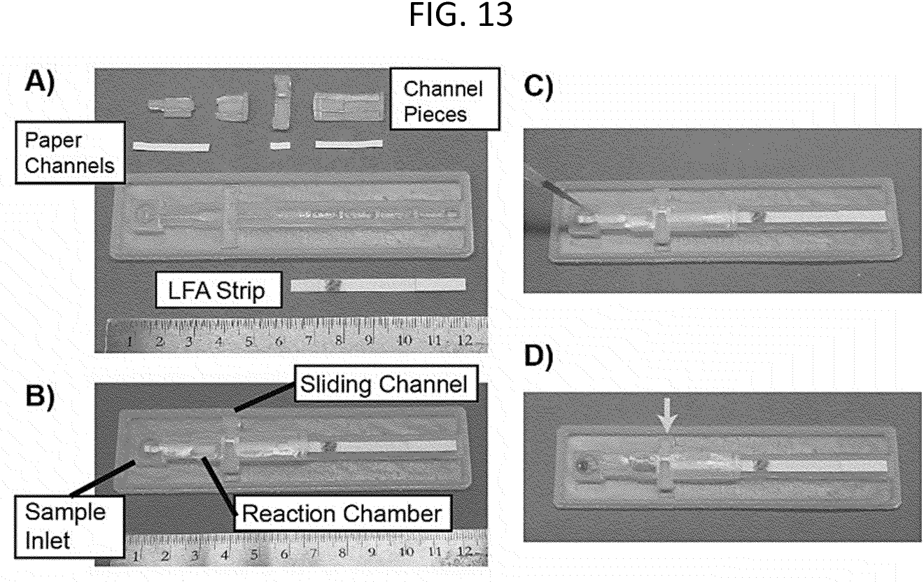

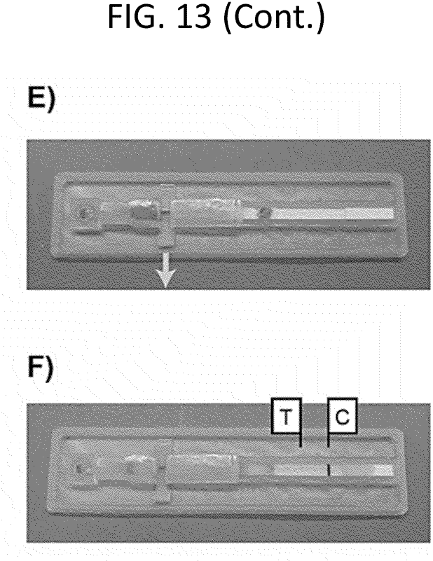

An exemplary diagnostic device and its use is illustrated in FIG. 13. The pieces of the device may be fabricated using SLA 3D printing (Form2, FormLabs), although those of skill in the art can prepare the device and components using other methods. Lysed sample is added to the inlet (FIG. 13C). As shown, the lysed sample is added via micropipette, but the lysed sample may be drained directly from a pierce-bottom sample tube, removing the need for liquid handling equipment. Lysate is wicked along the paper microchannel into the reaction chamber. Once the sample wicks into the reaction chamber, reaction reagents are mixed in, setting up the isothermal amplification (FIG. 13D). The reaction reagents may be lyophilized and stored within the reaction chamber when packaged until ready for use. The dry reagents will mix with the sample lysate following the addition of rehydration buffer, which will drain from a reagent blister reservoir.

Following RT-LAMP isothermal amplification, the sliding channel is moved from the inactive position (far end of its slot) to the active position (near end of its slot), providing fluidic connection between the reaction chamber and the LFA strip (FIG. 13E). The channel may be manually moved or automatically moved with the use of a controller. The amplicons wick through the sliding channel into the secondary paper microchannel all the way to the sample pad of the LFA strip. From there the sample wicks through the diagnostic strip, providing either a positive or negative readout (FIG. 13F).

### Example 8. Primers for Detection of the Delta Variant of SARS-CoV-2

Compared to the alpha variant of SARS-CoV-2, the genome of the delta variant comprises a single base pair mutation (from C to G) in a region corresponding to the 3′ end of the FIP primer. Hence, a new FIP primer comprising the nucleotide base “G” instead of “C” at the 3′ end was made for detection of delta variant RNA. The full primer set for the detection of this variant is provided in Table 24, below. In this primer set, the FIP primer comprises two subsequences: a F1c sequence comprising TAGGCAATGATGGATTGACTAGCTA (SEQ ID NO: 14) and a F2 sequence comprising TTATCAGACTCAGACTAATTCTCG (SEQ ID NO: 72) with a spacer therebetween. Likewise, the BIP primer comprises two subsequences: a B1c sequence comprising AACTCTATTGCCATACCCACAAAT (SEQ ID NO: 16) and a B2 sequence comprising TTGGTCATAGACACTGGTAG (SEQ ID NO: 17) with a spacer therebetween.

TABLE 24

RT-LAMP primer set for SARS-CoV-2 delta variant detection

| | |
|---|---|
| Name | Sequence (5′ to 3′) |
| FIP | TAGGCAATGATGGATTGACTAGCTATTTTTTATCAGACTCAGACTAATTCTCG (SEQ ID NO: 71) |
| BIP | AACTCTATTGCCATACCCACAAATTTTTTTGGTCATAGACACTGGTAG (SEQ ID NO: 21) |
| F3 | TTGGTGCAGGTATATGCG (SEQ ID NO: 12) |
| B3 | ACATTGTACAATCTACTGATGTC (SEQ ID NO: 13) |
| LF | CACTACGTGCCCGCCGA (SEQ ID NO: 18) |
| LB | TTTACTATTAGTGTTACC (SEQ ID NO: 19) |

To test the ability of this primer set to detect the delta variant, various concentrations of delta variant RNA (Twist Synthetic SARS-CoV-2 RNA Ctrl 23 1 (EPI_ISL_1544014); Synthetic RNA control (104533)) were spiked into 100 µl of SARS-COV-2 negative nasopharyngeal swab, mixed with 20 ul of inactivation buffer, and incubated at 95° C. for 5 minutes. A volume of 1 ml dilution buffer was then added to the lysed nasopharyngeal swab and mixed. A volume of 20 µl diluted nasopharyngeal sample was added to a 30 µl RT-LAMP reaction buffer made with the delta variant primer set and incubated at 65° C. for 25 minutes. The amplification results were read with the BioUSTAR EasyNAT. The results indicate that the delta primer set can detect delta variant RNA at concentrations as low as 222 copies/µl in nasopharyngeal swab samples.

What is claimed:

1. A composition for loop-mediated isothermal amplification of a target nucleotide sequence of SARS-CoV-2, the composition comprising:
a) a F3 primer comprising nucleotide sequence GATTTTTGTGGAAAGGGCTATC (SEQ ID NO: 1) or TTTTTGTGGAAAGGGCTATC (SEQ ID NO: 2) (5′ - 3′),
a B3 primer comprising nucleotide sequence CAAACCAGTGTGTGCCAT (SEQ ID NO: 3) (5′ - 3′);
a FIP primer comprising a F1c nucleotide sequence AGGGACATAAGTCACATGCAAGAA (SEQ ID NO: 4), a F2 nucleotide sequence TTCTTATGTCCTTCCCTCAGT (SEQ ID NO: 5), and a spacer therebetween (5′ - 3′); and
a BIP primer comprising a B1c nucleotide sequence AGAAAAGAACTTCACAACTGCTCC (SEQ ID NO: 6), a B2 nucleotide sequence CAAAGACACCTTCACGAGG (SEQ ID NO: 7), and a spacer therebetween (5′ - 3′);
b) a F3 primer comprising nucleotide sequence TTGGTGCAGGTATATGCG (SEQ ID NO: 12) (5′ - 3′);
a B3 primer comprising nucleotide sequence ACATTGTACAATCTACTGATGTC (SEQ ID NO: 13) (5′ - 3′);
a FIP primer comprising a F1c nucleotide sequence TAGGCAATGATGGATTGACTAGCTA (SEQ ID NO: 14), a F2 nucleotide sequence TTATCAGACTCAGACTAATTCTCC (SEQ ID NO: 15), and a spacer therebetween (5′ - 3′); and
a BIP primer comprising a B1c nucleotide sequence AACTCTATTGCCATACCCACAAAT (SEQ ID NO: 16), a B2 nucleotide sequence TTGGTCATAGACACTGGTAG (SEQ ID NO: 17), and a spacer therebetween (5′ - 3′); or
c) a F3 primer comprising nucleotide sequence TTGGTGCAGGTATATGCG (SEQ ID NO: 12) (5′ - 3′);
a B3 primer comprising nucleotide sequence ACATTGTACAATCTACTGATGTC (SEQ ID NO: 13) (5′ - 3′);
a FIP primer comprising a F1c nucleotide sequence TAGGCAATGATGGATTGACTAGCTA (SEQ ID NO: 14), a F2 nucleotide sequence TTATCAGACTCAGACTAATTCTCG (SEQ ID NO: 72), and a spacer therebetween (5′ - 3′); and
a BIP primer comprising a B1c nucleotide sequence AACTCTATTGCCATACCCACAAAT (SEQ ID NO: 16), a B2 nucleotide sequence TTGGTCATAGACACTGGTAG (SEQ ID NO: 17), and a spacer therebetween (5′ - 3′).

2. The composition of claim 1 further comprising:
a) a loop forward primer comprising a nucleotide sequence GACTACACCATGAGGTGCTG (SEQ ID NO: 8) (5′ - 3′) and a loop backward primer comprising a nucleotide sequence CATTTGTCATGATGGAAAAG (SEQ ID NO: 9) (5′ - 3′); or
b) a loop forward primer comprising a nucleotide sequence CACTACGTGCCCGCCGA (SEQ ID NO: 18) (5′ - 3′) and a loop backward primer comprising a nucleotide sequence TTTACTATTAGTGTTACC (SEQ ID NO: 19) (5′ - 3′).

3. The composition of claim 1, wherein the target nucleotide sequence is part of the sequence encoding the SARS-CoV-2 spike protein.

4. The composition of claim 3, wherein the target nucleotide sequence comprises SEQ ID NO: 69, SEQ ID NO: 70, or SEQ ID NO: 73.

5. The composition of claim 1, wherein one or more of the primers comprises a label.

6. The composition of claim 1 further comprising one or more of the following: deoxynucleotide triphosphates (dNTPs), a DNA polymerase, a reverse transcriptase, a buffering agent, primers for detecting a nucleotide control sequence, an enzyme co-factor, a positive control nucleic acid, a detergent, an inactivation buffer, a dilution buffer, a viral transport medium (VTM), and a dye.

7. A nucleic acid comprising, in order 5′ - 3′, the F1c, the F2, a nucleotide sequence substantially complementary to the F1c, the B1c, the B2, and a nucleotide sequence substantially complementary to the B1c according to claim 1.

8. The nucleic acid of claim 7, wherein the F1c and the nucleotide sequence substantially complementary to F1c hybridize and the B1c and the nucleotide sequence substantially complementary to B1c hybridize to form a dumbbell structure.

9. An amplification product of the nucleic acid of claim 7.

10. A method for detecting SARS-CoV-2 in a sample, the method comprising:
a) contacting the sample with the composition of claim 1 under conditions sufficient for loop-mediated isothermal amplification of the target nucleotide sequence of SARS-CoV-2; and
b) detecting the presence or absence of an amplification product of the target nucleotide sequence.

11. The method of claim 10, wherein RNA is extracted by lysis of the sample prior to the contacting step.

12. The method of claim 11, wherein the sample is treated with an RNase inactivation buffer prior to the contacting step.

13. The method of claim 11, wherein the extracted RNA is isolated or purified prior to the contacting step.

14. The method of claim 10 further comprising obtaining the sample from a subject having or suspected of having a SARS-CoV-2 infection.

15. The method of claim 14, wherein sample is a saliva sample, bronchoalveolar lavage, a sputum sample, a nasopharyngeal sample, a nasal sample, an oropharyngeal sample, sewage, or a stool sample.

16. The method of claim 10, wherein the amplification product is detected visually using:
a) a lateral flow device; or
b) a dye.

17. The method of claim 10, wherein the conditions sufficient for amplification of the target comprise an effective temperature of from 55-70° C. and/or an effective amplification time of between 20-60 minutes.

18. The method of claim 10, wherein the composition further comprises a composition for loop-mediated isothermal amplification of a control nucleotide sequence of the subject and the method further comprises contacting the composition with the sample under conditions sufficient for loop-mediated isothermal amplification of the control nucleotide sequence and detecting the presence or absence of an amplification product of the control nucleotide sequence.

19. A diagnostic device for detecting SARS-CoV-2, the diagnostic device comprising at least one reaction chamber comprising the composition of claim 1 and a detection device that provides a readout that indicates whether the target nucleic acid is present in the sample.

20. A kit comprising the composition of claim 1.


SEQUENCE LISTING (73 sequences)

SEQ ID NO: 1 - DNA, 22 bp, synthetic construct
  source 1..22: mol_type = other DNA
gatttttgtggaaagggctatc

SEQ ID NO: 2 - DNA, 20 bp, synthetic construct
  source 1..20: mol_type = other DNA
tttttgtggaaagggctatc

SEQ ID NO: 3 - DNA, 18 bp, synthetic construct
  source 1..18: mol_type = other DNA
caaaccagtgtgtgccat

SEQ ID NO: 4 - DNA, 24 bp, synthetic construct
  source 1..24: mol_type = other DNA
agggacataagtcacatgcaagaa

SEQ ID NO: 5 - DNA, 21 bp, synthetic construct
  source 1..21: mol_type = other DNA
ttcttatgtccttccctcagt

SEQ ID NO: 6 - DNA, 24 bp, synthetic construct
  source 1..24: mol_type = other DNA
agaaaagaacttcacaactgctcc

SEQ ID NO: 7 - DNA, 19 bp, synthetic construct
  source 1..19: mol_type = other DNA
caaagacaccttcacgagg

SEQ ID NO: 8 - DNA, 20 bp, synthetic construct
  source 1..20: mol_type = other DNA
gactacaccatgaggtgctg

SEQ ID NO: 9 - DNA, 20 bp, synthetic construct
  source 1..20: mol_type = other DNA
catttgtcatgatggaaaag

SEQ ID NO: 10 - DNA, 48 bp, synthetic construct
  source 1..48: mol_type = other DNA
agggacataagtcacatgcaagaatttttcttatgtccttccctcagt

SEQ ID NO: 11 - DNA, 47 bp, synthetic construct
  source 1..47: mol_type = other DNA
agaaaagaacttcacaactgctccttttcaaagacaccttcacgagg

SEQ ID NO: 12 - DNA, 18 bp, synthetic construct
  source 1..18: mol_type = other DNA
ttggtgcaggtatatgcg

SEQ ID NO: 13 - DNA, 23 bp, synthetic construct
  source 1..23: mol_type = other DNA
acattgtacaatctactgatgtc

SEQ ID NO: 14 - DNA, 25 bp, synthetic construct
  source 1..25: mol_type = other DNA
taggcaatgatggattgactagcta

SEQ ID NO: 15 - DNA, 24 bp, synthetic construct
  source 1..24: mol_type = other DNA
ttatcagactcagactaattctcc

SEQ ID NO: 16 - DNA, 24 bp, synthetic construct
  source 1..24: mol_type = other DNA
aactctattgccatacccacaaat

SEQ ID NO: 17 - DNA, 20 bp, synthetic construct
  source 1..20: mol_type = other DNA
ttggtcatagacactggtag

SEQ ID NO: 18 - DNA, 17 bp, synthetic construct
  source 1..17: mol_type = other DNA
cactacgtgcccgccga

SEQ ID NO: 19 - DNA, 18 bp, synthetic construct
  source 1..18: mol_type = other DNA
tttactattagtgttacc

SEQ ID NO: 20 - DNA, 53 bp, synthetic construct
  source 1..53: mol_type = other DNA
taggcaatgatggattgactagctattttttatcagactcagactaattctcc

SEQ ID NO: 21 - DNA, 48 bp, synthetic construct
  source 1..48: mol_type = other DNA
aactctattgccatacccacaaatttttttggtcatagacactggtag

SEQ ID NO: 22 - DNA, 22 bp, synthetic construct
  source 1..22: mol_type = other DNA
gttcagattctaaaacagtgct

SEQ ID NO: 23 - DNA, 20 bp, synthetic construct
  source 1..20: mol_type = other DNA
tgtccaatacagtagccatt

SEQ ID NO: 24 - DNA, 47 bp, synthetic construct
  source 1..47: mol_type = other DNA
agtgactaccttattggatagcccttttctcgtagagttttctgcgt

SEQ ID NO: 25 - DNA, 51 bp, synthetic construct
  source 1..51: mol_type = other DNA
tatgacaagtgcaactgagaaacattttgccacatgtgactatatacattc

SEQ ID NO: 26 - DNA, 21 bp, synthetic construct
  source 1..21: mol_type = other DNA
agatacagaatatcttcctca

SEQ ID NO: 27 - DNA, 20 bp, synthetic construct
  source 1..20: mol_type = other DNA
gaaacgaggggttactaatc

SEQ ID NO: 28 - DNA, 21 bp, synthetic construct
  source 1..21: mol_type = other DNA
acaaaaaaattaggcatggtg

SEQ ID NO: 29 - DNA, 19 bp, synthetic construct
  source 1..19: mol_type = other DNA
aaaccccacagatttcctt

SEQ ID NO: 30 - DNA, 45 bp, synthetic construct
  source 1..45: mol_type = other DNA
ctctcaggtccaaacgatcctcttttcacatgcctgtagtcctag

SEQ ID NO: 31 - DNA, 21 bp, synthetic construct
  source 1..21: mol_type = other DNA
acaaaaaaattaggcatggtg

SEQ ID NO: 32 - DNA, 20 bp, synthetic construct
  source 1..20: mol_type = other DNA
ggcatacaatttgcttgctt

SEQ ID NO: 33 - DNA, 22 bp, synthetic construct
  source 1..22: mol_type = other DNA
atcatgtatgggtataggttct

SEQ ID NO: 34 - DNA, 51 bp, synthetic construct
  source 1..51: mol_type = other DNA
cgcaaagagaatggtagttccctaattttgtgtgtgcatagatctactgac

SEQ ID NO: 35 - DNA, 50 bp, synthetic construct
  source 1..50: mol_type = other DNA
ggaagccacatatgcctatctaggtttttcttcttcaccattatccagaa

SEQ ID NO: 36 - DNA, 20 bp, synthetic construct
  source 1..20: mol_type = other DNA
gcttaatctatgtgtgtgca

SEQ ID NO: 37 - DNA, 22 bp, synthetic construct
  source 1..22: mol_type = other DNA
atcatgtatgggtataggttct

SEQ ID NO: 38 - DNA, 46 bp, synthetic construct
  source 1..46: mol_type = other DNA
gcttcctacgcaaagagaatggtattttagatctactgacacacgc

SEQ ID NO: 39 - DNA, 51 bp, synthetic construct
  source 1..51: mol_type = other DNA
atatgcctatctaggcctcagatcttttatcttcttcaccattatccagaa

SEQ ID NO: 40 - DNA, 19 bp, synthetic construct
  source 1..19: mol_type = other DNA
tctactgacacacgcatac

SEQ ID NO: 41 - DNA, 18 bp, synthetic construct
  source 1..18: mol_type = other DNA
acaggttgctacgctaga

SEQ ID NO: 42 - DNA, 52 bp, synthetic construct
  source 1..52: mol_type = other DNA
tatgatctgaggcctagataggcattttattagggaactaccattctctttg

SEQ ID NO: 43 - DNA, 51 bp, synthetic construct
  source 1..51: mol_type = other DNA
gaataggctttctggataatggtgattttatcatgtatgggtataggttct

SEQ ID NO: 44 - DNA, 18 bp, synthetic construct
  source 1..18: mol_type = other DNA
atctagaggctgggaagg

SEQ ID NO: 45 - DNA, 19 bp, synthetic construct
  source 1..19: mol_type = other DNA
agtgtcctttccagaaact

SEQ ID NO: 46 - DNA, 44 bp, synthetic construct
  source 1..44: mol_type = other DNA
cctatggcttggactttccaacttttgctcctgaaccagttgtt

SEQ ID NO: 47 - DNA, 46 bp, synthetic construct
  source 1..46: mol_type = other DNA
agcttacgttatctaccagagcattttggtataaatgcgaaccgct

SEQ ID NO: 48 - DNA, 20 bp, synthetic construct
  source 1..20: mol_type = other DNA
gcagacatacaacggacggt

SEQ ID NO: 49 - DNA, 16 bp, synthetic construct
  source 1..16: mol_type = other DNA
acctacccagccgcag

SEQ ID NO: 50 - DNA, 45 bp, synthetic construct
  source 1..45: mol_type = other DNA
cgttagtgggtgacctaggcacttttccagacccaggctgtgtag

SEQ ID NO: 51 - DNA, 43 bp, synthetic construct
  source 1..43: mol_type = other DNA
accctcaaaagcaggcagctttttggaaggaaggtgggctcta

SEQ ID NO: 52 - DNA, 48 bp, synthetic construct; identical to SEQ ID NO: 10
  source 1..48: mol_type = other DNA

SEQ ID NO: 53 - DNA, 47 bp, synthetic construct; identical to SEQ ID NO: 11
  source 1..47: mol_type = other DNA

SEQ ID NO: 54 - DNA, 22 bp, synthetic construct
  source 1..22: mol_type = other DNA
gatttttgtggaaagggctatc

SEQ ID NO: 55 - DNA, 18 bp, synthetic construct
  source 1..18: mol_type = other DNA
caaaccagtgtgtgccat

SEQ ID NO: 56 - DNA, 20 bp, synthetic construct
  source 1..20: mol_type = other DNA
gactacaccatgaggtgctg

SEQ ID NO: 57 - DNA, 20 bp, synthetic construct
  source 1..20: mol_type = other DNA
catttgtcatgatggaaaag

SEQ ID NO: 58 - DNA, 53 bp, synthetic construct; identical to SEQ ID NO: 20
  source 1..53: mol_type = other DNA

SEQ ID NO: 59 - DNA, 48 bp, synthetic construct; identical to SEQ ID NO: 21
  source 1..48: mol_type = other DNA

SEQ ID NO: 60 - DNA, 18 bp, synthetic construct
  source 1..18: mol_type = other DNA
ttggtgcaggtatatgcg

SEQ ID NO: 61 - DNA, 23 bp, synthetic construct
  source 1..23: mol_type = other DNA
acattgtacaatctactgatgtc

SEQ ID NO: 62 - DNA, 17 bp, synthetic construct
  source 1..17: mol_type = other DNA
cactacgtgcccgccga

SEQ ID NO: 63 - DNA, 18 bp, synthetic construct
  source 1..18: mol_type = other DNA
tttactattagtgttacc

SEQ ID NO: 64 - DNA, 44 bp, synthetic construct; identical to SEQ ID NO: 46
  source 1..44: mol_type = other DNA

SEQ ID NO: 65 - DNA, 46 bp, synthetic construct; identical to SEQ ID NO: 47
  source 1..46: mol_type = other DNA

SEQ ID NO: 66 - DNA, 18 bp, synthetic construct
  source 1..18: mol_type = other DNA
atctagaggctgggaagg

SEQ ID NO: 67 - DNA, 19 bp, synthetic construct
  source 1..19: mol_type = other DNA
agtgtcctttccagaaact

SEQ ID NO: 68 - DNA, 22 bp, synthetic construct
  source 1..22: mol_type = other DNA
cctgacagaccgacaagacgga

SEQ ID NO: 69 - RNA, 190 nt, synthetic construct
  source 1..190: mol_type = other RNA
gatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttg

SEQ ID NO: 70 - RNA, 225 nt, synthetic construct
  source 1..225: mol_type = other RNA
ttggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgt

SEQ ID NO: 71 - DNA, 53 bp, synthetic construct
  source 1..53: mol_type = other DNA
taggcaatgatggattgactagctattttttatcagactcagactaattctcg

SEQ ID NO: 72 - DNA, 24 bp, synthetic construct
  source 1..24: mol_type = other DNA
ttatcagactcagactaattctcg

SEQ ID NO: 73 - RNA, 225 nt, synthetic construct
  source 1..225: mol_type = other RNA
ttggtgcaggtatatgcgctagttatcagactcagactaattctcgtcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgt